(19) United States
(12) Patent Application Publication
(10) Pub. No.: US 2026/0045036 A1
(43) Pub. Date: Feb. 12, 2026

(54) Methods and Systems for Generating Surface Models of Cardiac Structures

(71) Applicant: St. Jude Medical, Cardiology Division, Inc., St. Paul, MN (US)

(72) Inventor: Eric S. Olson, Maplewood, MN (US)

(73) Assignee: St. Jude Medical, Cardiology Division, Inc., St. Paul, MN (US)

(21) Appl. No.: 19/295,934

(22) Filed: Aug. 11, 2025

Related U.S. Application Data
(60) Provisional application No. 63/681,641, filed on Aug. 9, 2024.

(51) Int. Cl. G06T 17/00; A61B 5/367; A61M 25/01; G06T 19/20

(52) U.S. Cl. CPC G06T 17/00; A61M 25/0127; G06T 19/20; A61B 5/367; A61M 2025/0166; G06T 2210/41; G06T 2210/56; G06T 2219/2016

(57) ABSTRACT

A system for generating a surface model of a cardiac structure includes a display device, a medical device, and a model reconstruction system. The medical device includes one or more sensors configured to generate sensor output used to generate location data for points disposed on a surface of the cardiac structure. The model reconstruction system configured to: (a) process the sensor output to generate a point cloud that represents measured locations on the surface of the cardiac structure and/or within the cardiac structure, (b) extract a surface point cloud from the point cloud, (d) generate a final signed distance field (SDF) representing a shape of a surface of the cardiac structure via a machine learning model, (e) construct a surface model representing the shape of the surface of the cardiac structure based on the final SDF, and output or display the surface model to a user via the display device.

### CROSS-REFERENCES TO RELATED APPLICATIONS

The present application claims the benefit of U.S. Provisional Application No. 63/681,641 filed Aug. 9, 2024, the full disclosure which is incorporated herein by reference in its entirety for all purposes.

### BACKGROUND

The generation of surface models from point clouds generated via tracking the location of catheters disposed in the heart is a common task required during electrophysiology mapping procedures. Surface model generation has progressed over the decades using techniques spanning 3D convex hulls up to alpha shapes and voxel-based methods. The latter methods perform well when the point cloud is dense and spans the entire geometry. However, this can be a time-consuming process that takes upwards of 20 minutes. Further, if the operator fails to densely sample the entire chamber, the resulting surface model may not accurately represent the actual chamber shape. This can happen if the data is sparse or if portions of the data are missing resulting in gaps in the point cloud (voids). Using present techniques, the resulting surface model may be missing regions corresponding to the voids in the data. Thus, improvements to systems and methods for generating surface models of cardiac structures are desired.

### BRIEF SUMMARY

Systems and computer-implemented methods for generating surface models of cardiac structures are described. These systems and computer-implemented methods can be of particular benefit in the case of sparse or absent data characterizing the cardiac structure of the patient's heart. Specifically, in some systems, devices, and computer-implemented methods of the present disclosure, data characterizing the cardiac structure can be gathered as part of a procedure. However, the gathering of this data can be time consuming and challenging. The data gathered may be insufficient to accurately characterize the cardiac structure using previously known techniques. However, the presently disclosed systems and computer-implemented methods can leverage this sparse data, in combination with a specifically trained neural network, to automatically generate an accurate surface model of the cardiac structure, thereby mitigating the problems created by the sparse data.

Thus, in one aspect, a first computer-implemented method of generating a surface model of a cardiac structure of a patient's heart is provided. The first computer-implemented method includes: (a) receiving, one or several processors, a point cloud comprising a plurality of points, wherein each of the points represents a measured location on a surface of the cardiac structure; (b) extracting a surface point cloud from the point cloud, wherein the surface point cloud comprises points that represent locations on the surface of the cardiac structure; (c) generating a final signed distance field (SDF) representing the surface of the cardiac structure via a machine learning model; (d) constructing a faceted surface representing the surface of the cardiac structure based on the final SDF; and (e) outputting the faceted surface to a user via a graphical user interface.

In the first computer-implemented method, the faceted surface can have any suitable configuration. For example, the faceted surface comprises a triangulated surface.

In the first computer-implemented method, contact information associated with each of the points in the point cloud can be employed. The contact information can be associated with each of the points in the point cloud and indicate whether the represented location is on the surface of the cardiac structure. The contact information can be generated based on at least one of: (a) a contact force detected by a device used to measure locations used to generate the point cloud; or (b) a complex impedance sensed by the device. The points can be extracted from the point cloud based on the contact information.

The first computer-implemented method can further include defining an approximating surface that approximates the surface of the cardiac structure. The points can be extracted from the point cloud based on proximity of the points to the approximating surface.

In the first computer-implemented method, the machine learning model can have any suitable configuration. For example, the machine learning model can include an autodecoder.

In the first computer-implemented method, the generation of the final SDF can be accomplished using any suitable approach. For example, the generation of the final SDF can include: (a) selecting an input vector for ingestion into the machine learning model, and (b) determining an error between a preliminary SDF generated via the machine learning model and the surface point cloud. The generation of the final SDF can include optimizing the input vector to minimize the error between the final SDF and the surface point cloud. The optimization of the input vector to minimize the error between the final SDF and the surface point cloud can include optimizing the input vector via a gradient descent algorithm. The gradient descent algorithm can include an Adaptive Moment Estimation (ADAM) optimizer.

The first computer-implemented method can include optimizing an affine transformation and using the optimized affine transformation to compensate for an orientation of the patient's heart. The optimization of the affine transformation used to compensate for the orientation of the patient's heart can be performed as part of optimizing the input vector to minimize the error between the final SDF and the surface point cloud.

The first computer-implemented method can include generating the final SDF from the preliminary SDF by upscaling the preliminary SDF. The preliminary SDF can be upscaled to the final SDF via application of a scale factor to the preliminary SDF.

In the first computer-implemented method, any suitable approach can be used to construct the faceted surface representing the surface of the cardiac structure based on the final SDF. For example, the construction of the faceted surface representing the surface of the cardiac structure based on the final SDF can include: (a) converting the final SDF into a voxel format, and (b) converting the voxel format into the faceted surface. The construction of the faceted surface representing the surface of the cardiac structure based on the final SDF can include: (a) creating a final point cloud from the final SDF, and (b) converting the final point cloud into the faceted surface.

In the first computer-implemented method, any suitable approach can be used to convert the final point cloud into the faceted surface. For example, the final point cloud can be converted into the faceted surface via application of at least one of: (a) an alpha shape algorithm, (b) a ball pivoting algorithm, and (c) a Poisson surface reconstruction.

In another aspect, a tangible computer-readable storage medium stores instructions executable by the one or several processors to cause the one or several processors to accomplish the first computer-implemented method, as well as any of the variations thereof in accordance with the present disclosure.

In another aspect, a second computer-implemented method of generating a surface model of a cardiac structure of a patient's heart is provided. The second computer-implemented method includes: (a) receiving, by at least one processor, one or more intracardiac images of the cardiac structure; (b) processing the one or more intracardiac images to generate a surface point cloud comprising points that represent locations on a surface of the cardiac structure; (c) generating a final signed distance field (SDF) representing the surface of the cardiac structure via a machine learning model; (d) constructing a faceted surface representing the surface of the cardiac structure based on the final SDF; and (e) outputting the faceted surface to a user via a graphical user interface.

In the second computer-implemented method, the faceted surface can have any suitable configuration. For example, the surface model can include a faceted surface.

In the second computer-implemented method, the one or more intracardiac images can be generated using any suitable approach. For example, the one or more intracardiac images are generated via intracardiac echocardiography.

In the second computer-implemented method, the processing of the one or more intracardiac images to generate the surface point cloud can be accomplished using any suitable approach. For example, the processing of the one or more intracardiac images to generate the surface point cloud comprises one or more of: (a) identifying a contour of the cardiac structure in the one or more intracardiac images and generating points representing locations on the identified contour of the cardiac structure; or (b) identifying a surface of the cardiac structure in the one or more intracardiac images and generating points representing locations on the identified surface of the cardiac structure.

In the second computer-implemented method, the machine learning model can have any suitable configuration. For example, the machine learning model can include an autodecoder.

In the second computer-implemented method, the final SDF can be generated using any suitable approach. For example, the generation of the final SDF can include: (a) selecting an input vector for ingestion into the machine learning model; and (b) determining an error between a preliminary SDF generated via the machine learning model and at least some of the points in the surface point cloud. The generation of the final SDF can include optimizing the input vector to minimize the error between the final SDF and the at least some of the points in the surface point cloud. The optimization of the input vector to minimize the error between the final SDF and the at least some of the points in the surface point cloud can include optimizing the input vector via a gradient descent algorithm. The gradient descent algorithm can include an Adaptive Moment Estimation (ADAM) optimizer.

The second computer-implemented method can include optimizing an affine transformation and using the optimized affine transformation to compensate for an orientation of the patient's heart. The optimization of the affine transformation can be performed as part of optimizing the input vector to minimize the error between the final SDF and the at least some of the points in the surface point cloud.

In the second computer-implemented method, the preliminary SDF can be normalized. The second computer-implemented method can include generating the final SDF from the preliminary SDF by upscaling the preliminary SDF. The preliminary SDF can be upscaled to the final SDF via application of a scale factor to the preliminary SDF.

In the second computer-implemented method, the construction of the surface model representing the surface of the cardiac structure based on the final SDF can be accomplished using any suitable approach. For example, the construction of the surface model representing the surface of the cardiac structure based on the final SDF can include: (a) converting the final SDF into a voxel format, and (b) converting the voxel format into the surface model. The construction of the surface model representing the surface of the cardiac structure based on the final SDF can include: (a) creating a final point cloud from the final SDF, and (b) generating the surface model based on the final point cloud. The generation of the surface model based on the final point cloud can be accomplished via application of at least one of: (a) an alpha shape algorithm, (b) a ball pivoting algorithm, and (c) a Poisson surface reconstruction.

In another aspect, a tangible computer-readable storage medium stores instructions executable by the one or several processors to cause the one or several processors to accomplish the second computer-implemented method, as well as any of the variations thereof in accordance with the present disclosure.

In another aspect, a first system for generating a surface model of a cardiac structure of a patient's heart includes a display device, a medical device, and a model reconstruction system. The medical device includes one or more sensors configured to generate sensor output used to generate location data for points disposed on a surface of the cardiac structure and/or disposed within the cardiac structure. The model reconstruction system includes one or several processors and a tangible memory storing non-transient instructions executable by the one or several processors to cause the one or several processors to: (a) process the sensor output to generate a point cloud comprising a plurality of points, wherein each of the points represents a measured location on the surface of the cardiac structure and/or within the cardiac structure; (b) extract a surface point cloud from the point cloud, wherein the surface point cloud comprises points that represent measured locations on the surface of the cardiac structure; (c) generate a final signed distance field (SDF) representing a shape of a surface of the cardiac structure based at least in part of the surface point cloud via a machine learning model; (d) construct a surface model representing the shape of the surface of the cardiac structure based on the final SDF; and (e) output or display the surface model to a user via the display device.

In the first system for generating a surface model of a cardiac structure, the surface model can have any suitable configuration. For example, the surface model can include a faceted surface.

In the first system for generating a surface model of a cardiac structure, contact information can be associated with each of the points in the point cloud and can indicate whether the point represents a location on the surface of the cardiac structure. The contact information can be generated based on at least one of: (a) a contact force detected by the medical device used to generate the sensor output used to generate the location data for the points that represent measured locations on the surface of the cardiac structure; or (b) a complex impedance sensed by the medical device. The surface point cloud can be extracted from the point cloud based on the contact information.

In the first system for generating a surface model of a cardiac structure, the non-transient instructions can be further executable by the one or several processors to cause the one or several processors to define an approximating surface that approximates the surface of the cardiac structure. The surface point cloud can be extracted from the point cloud based on proximity of the points to the approximating surface.

In the first system for generating a surface model of a cardiac structure, the machine learning model can have any suitable configuration. For example, the machine learning model can include an autodecoder.

In the first system for generating a surface model of a cardiac structure, the final SDF can be generated using any suitable approach. For example, the generation of the final SDF can include: (a) selecting an input vector for ingestion into the machine learning model; and (b) determining an error between a preliminary SDF generated via the machine learning model and the surface point cloud. The generation of the final SDF can include optimizing the input vector to minimize the error between the final SDF and the surface point cloud. The optimization of the input vector to minimize the error between the final SDF and the surface point cloud can include optimizing the input vector via a gradient descent algorithm. The gradient descent algorithm can include an Adaptive Moment Estimation (ADAM) optimizer.

In the first system for generating a surface model of a cardiac structure, the non-transient instructions can be further executable by the one or several processors to compensate for an orientation of the patient's heart. For example, the non-transient instructions can be further executable by the one or several processors to cause the one or several processors to optimize an affine transformation used to compensate for an orientation of the patient's heart. The optimization of the affine transformation used to compensate for the orientation of the patient's heart can be performed as part of optimizing the input vector to minimize the error between the final SDF and the surface point cloud.

In the first system for generating a surface model of a cardiac structure, the final SDF is generated from the preliminary SDF by upscaling the preliminary SDF. The preliminary SDF can be up scaled to the final SDF via application of a scale factor to the preliminary SDF.

In the first system for generating a surface model of a cardiac structure, the construction of the surface model representing the shape of the surface of the cardiac structure based on the final SDF can be accomplished using any suitable approach. For example, the construction of the surface model representing the shape of the surface of the cardiac structure based on the final SDF can include: (a) converting the final SDF into a voxel format; and (b) converting the voxel format into the surface model. The construction of the surface model representing the shape of the surface of the cardiac structure based on the final SDF can include: (a) creating a final point cloud from the final SDF; and (b) converting the final point cloud into the surface model. The final point cloud can be converted into the surface model via application of at least one of: (a) an alpha shape algorithm; (b) a ball pivoting algorithm; and (c) a Poisson surface reconstruction.

In another aspect, a second system for generating a surface model of a cardiac structure of a patient's heart includes a display device, a medical device, and a model reconstruction system. The medical device includes one or more image sensors (182) configured to generate image sensor output used to generate surface data that defines a surface of the cardiac structure. The model reconstruction system includes one or several processors and a tangible memory storing non-transient instructions executable by the one or several processors to cause the one or several processors to: (a) process the image sensor output to generate the surface data that defines the surface of the cardiac structure, (b) process the surface data to generate a surface point cloud comprising points representing locations on the surface of the cardiac structure, (c) generate a final signed distance field (SDF) representing a shape of a surface of the cardiac structure based on the surface point cloud via a machine learning model, (d) construct a surface model representing the shape of the surface of the cardiac structure based on the final SDF, and (e) output or display the surface model to a user via the display device.

In the second system for generating a surface model of a cardiac structure, the surface model can have any suitable configuration. For example, the surface model can include a faceted surface.

In the second system for generating a surface model of a cardiac structure, the image sensor output can be generated using any suitable approach. For example, the image sensor output can be generated via intracardiac echocardiography.

In the second system for generating a surface model of a cardiac structure, the surface data that defines the surface of the cardiac structure can include any suitable data. For example, the surface data that defines the surface of the cardiac structure comprises at least one of: (a) a contour extracted from one or more 2-dimensional images of the cardiac structure; (b) a surface extracted from one or more 3-dimensional images of the cardiac structure; or (c) a point on a contour extracted from one or more 2-dimensional images of the cardiac structure.

In the second system for generating a surface model of a cardiac structure, the machine learning model can have any suitable configuration. For example, the machine learning model can include an autodecoder.

In the second system for generating a surface model of a cardiac structure, the final SDF can be generated using any suitable approach. For example, the generation of the final SDF can include: (a) selecting an input vector for ingestion into the machine learning model, and (b) determining an error between a preliminary SDF generated via the machine learning model and at least some of the points in the surface point cloud. The generation of the final SDF can include optimizing the input vector to minimize the error between the final SDF and the at least some of the points in the surface point cloud. The optimization of the input vector to minimize the error between the final SDF and the at least some of the points in the surface point cloud can include optimizing the input vector via a gradient descent algorithm. The gradient descent algorithm can include an Adaptive Moment Estimation (ADAM) optimizer.

In the second system for generating a surface model of a cardiac structure, the non-transient instructions can be further executable by the one or several processors to cause the one or several processors to compensate for an orientation of the patient's heart. For example, the non-transient instructions can be further executable by the one or several processors to cause the one or several processors to optimize an affine transformation used to compensate for an orientation of the patient's heart. The optimization of the affine transformation can be performed as part of optimizing the input vector to minimize the error between the final SDF and the at least some of the points in the surface point cloud.

In the second system for generating a surface model of a cardiac structure, the preliminary SDF can be normalized and the non-transient instructions can be further executable by the one or several processors to cause the one or several processors to generate the final SDF from the preliminary SDF by upscaling the preliminary SDF. The preliminary SDF can be upscaled to the final SDF via application of a scale factor to the preliminary SDF.

In the second system for generating a surface model of a cardiac structure, the surface model representing the surface of the cardiac structure can be constructed based on the final SDF using any suitable approach. For example, the construction of the surface model representing the surface of the cardiac structure based on the final SDF can include: (a) converting the final SDF into voxel format, and (b) converting the voxel format into the surface model. The construction of the surface model representing the shape of the surface of the cardiac structure based on the final SDF can include: (a) creating a final point cloud from the final SDF, and (b) generating the surface model based on the final point cloud. The final point cloud can be converted into the surface model via application of at least one of: (a) an alpha shape algorithm, (b) a ball pivoting algorithm, and (c) a Poisson surface reconstruction.

### BRIEF DESCRIPTION OF THE DRAWINGS

The accompanying drawings, which are incorporated in and constitute a part of the specification, illustrate one or more systems, devices, and computer-implemented methods of the present disclosure and, together with the description, explain the systems, devices, and computer-implemented methods of the present disclosure. The accompanying drawings have not necessarily been drawn to scale. Any values dimensions illustrated in the accompanying graphs and figures are for illustration purposes only and may or may not represent actual or preferred values or dimensions. Where applicable, some or all features cannot be illustrated to assist in the description of underlying features.

FIG. 1 depicts one example of a high-level system for generating surface models of cardiac structures.

FIG. 2 depicts a detailed example of a system for generating surface models of cardiac structures.

FIG. 3 depicts portions of a system for generating surface models of cardiac structures.

FIG. 4 is a schematic depiction of examples of a process for generating surface models of cardiac structures.

FIG. 5 is a flowchart illustrating a process for generating a surface model of a cardiac structure.

FIG. 6 is a flowchart illustrating a process for generating a surface model of a cardiac structure using an imaging device.

FIG. 7 is a flowchart illustrating one example of a process for generation of a final SDF.

FIG. 8 is a flowchart illustrating another example of a process for generation of a final SDF.

FIG. 9 is a flowchart illustrating an example of a process for generating a surface model of a cardiac structure based on a final SDF via a voxel representation.

FIG. 10 a flowchart illustrating one example of a process for generating a surface model of a cardiac structure based on a final SDF via a final point cloud.

FIG. 11 is a schematic depiction of examples of a process for training a machine learning model for generating surface models of cardiac structures.

### DETAILED DESCRIPTION

The description set forth below in connection with the appended drawings is intended as a description of various examples of the disclosed subject matter and is not necessarily intended to represent the only systems, devises, and computer-implemented methods within the scope of the present disclosure. In certain instances, the description includes specific details for the purpose of providing an understanding of the disclosed systems, devices, and computer-implemented methods of the present disclosure. However, it will be apparent to those skilled in the art that the disclosed systems, devices, and computer-implemented methods of the present disclosure can be practiced without those specific details. In some instances, well-known structures and components can be shown in block diagram form to avoid obscuring the concepts of the disclosed subject matter.

Cardiac Electrophysiology (EP) relies on the virtual generation of surface models of portions of the heart to facilitate in determining properties of those portions of the heart, in identifying locations of those portions of the heart for receiving a treatment, for delivering treatment to those portions of the heart, and/or for evaluating the efficacy of the delivered treatment. Generation surface models from point clouds generated via tracking the location of catheters disposed in the heart is a common task during electrophysiology mapping procedures. Although generation of surface models of cardiac structures is common, it is not simple. For example, an operator must take great care to properly sample the portions of the heart for which surface models are being constructed. If this sampling is not adequate, for example if the operator fails to densely sample the entire chamber, the resulting surface model may not represent the actual chamber shape.

The present disclosure relates to systems and computer-implemented methods for generating surface models of cardiac structures. Generation of surface models of cardiac structures as described herein improves over current techniques as the generation described herein can accurately generate surface models of cardiac structures while significantly reducing the burden on the operator to densely sample the anatomy for which the surface models are being generated. Thus, the present disclosure addresses sparse sampling and/or problems arising from sparse sampling to thereby provide a complete and accurate representation of the represented anatomy.

In contrast to previous techniques, application of the present disclosure can, advantageously, provide “shape completion” when the sampling of the cardiac structure is sparse or incomplete. Whereas prior methods would be unable to create accurate surface models from such data, the present disclosure utilizes, in combination with a machine learning model, such as a neural network, statistical shape prior information to produce an accurate surface model of the cardiac structure from far less data. In other words, utilizing a machine learning model such as a neural network, the present disclosure generates surface models that accurately represent patient anatomy even when the sampling data for the cardiac structure is sparse or incomplete. This allows the electrophysiologist to create the surface model of the cardiac structure in notably less time as the electrophysiologist no longer must capture such dense data to accurately represent the target anatomy, and specifically to accurately represent the cardiac anatomy. Further, this allows accurate representation of patient anatomy even when sampled data is sparse or includes missing portions of data, or in other words even when the sampled data does not cover all portions of the heart for which surface models are being generated.

These benefits may be achieved via an algorithm that creates an ingestible input from received data characterizing an aspect of a patient's anatomy such as all or portions of a patient's heart. This received data can be a point cloud such as a georeferenced point cloud, also referred to herein as a GEO point cloud, or the received data can be contours or surfaces received from imaging, such as endocardial contours or surfaces captured via ultrasound or CT/MRI imaging. This ingestible input is then used in an iterative process whereby a final signed distance field (SDF) representing a shape of at least a portion of the patient's anatomy is generated. This final SDF can be generated via a machine learning model and based at least in part on the ingestible input and/or the received data. Via the utilization of the machine learning model to generate the final SDF, the detrimental effects of a sparse data set can be eliminated and/or mitigated.

The final SDF can be utilized to construct a surface representation. This can include the generation of a faceted surface based on the final SDF. The faceted surface can be a polygon mesh and specifically can be a triangulated mesh. This faceted surface can be outputted to a user via a graphical user interface.

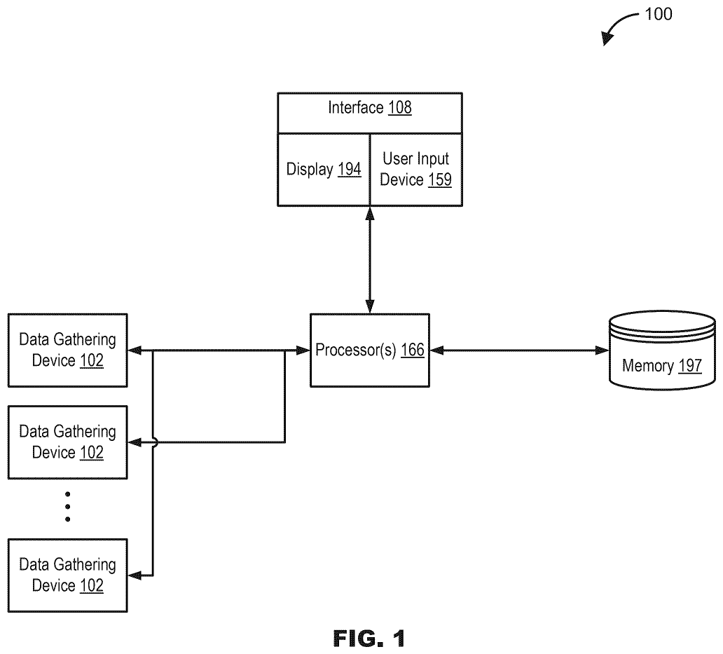

FIG. 1 illustrates a high-level system 100 for generating surface models of cardiac structures. The system 100 can gather information from the patient that is used in generating the surface models of the cardiac structure.

The system 100 includes one or several data gathering devices 102. Each of the one or several data gathering devices 102 can be configured to gather patient data and specifically can be configured to gather patient data inside of the patient from inside of the patient and/or from outside of the patient. These one or several data gathering devices 102 can include, for example, a catheter that can include one or several sensors, electrodes, or the like. The one or several data gathering devices 102 can include, for example, one or several imaging devices configured to generate images within the patient and/or of within the patient such as, for example, one or several echocardiographs and specifically one or several intracardiac echocardiographs, one or several cameras, or the like. The one or several data gathering devices 102 can be configured to generate one or several points corresponding to locations within the patient's heart and/or within a chamber of the patient's heart and/or to capture images of one or several surfaces within the patient's heart and/or within a chamber of the patient's heart. The patient data can be data gathered inside of the patient from outside of the patient with equipment, one or several devices, one or several sensors, and/or one or several tools external to the patient such as, for example, a CT and/or MRI.

The system 100 includes one or several processors 166, also referred to herein as a processing apparatus 166. The one or several processors 166 can be configured to receive and/or process data, to generate control signals controlling operation of one or several components of the system 100, and/or to train and/or utilize a machine learning model which can be a neural network. The one or several processors 166 can include one or several Central Processing Units (CPUs), one or several Graphics Processing Units (GPUs), one or several integrated circuits, one or several ASICs, one or several control units, or the like. The one or several processors 166 can be co-located with each other and/or with one or several of the other components of the system 100, can be located remote from the other components of the system 100, and/or can be provided as a cloud-based service.

The one or several processors 166 can be communicatively connected with the one or several data gathering devices 102 such that the one or several processors can control operation of the one or several data gathering devices 102 and/or can receive data and/or electrical signals from the one or several data gathering devices 102.

The one or several processors 166 can be further communicatively connected with a memory 197. The memory 197 can be configured to store computer executable instructions, data gathered by the one or several data gathering devices 102, one or several machine learning models and specifically one or several neural networks, data generated by the one or several machine learning models and specifically by the one or several neural networks, training data, or the like.

The memory 197 can be any desired memory including, for example, main memory, auxiliary memory, or the like. The memory 197 can include volatile memory and/or non-volatile memory. The memory 197 can be co-located with other components of the system 100, can be located remote from the other components of the system 100, and/or can be provided as a cloud-based service. The memory can store instructions executable by the one or several processors 166 to cause the one or several processors 166 to take one or several actions.

The system 100 can include interface 108. The interface 108 can be configured to provide information to the user and/or to receive inputs from the user. The interface 108 can include an output feature such as a speaker or a display 194, which can include one or several screens, touchscreens, or the like. The interface 108 can include a user input device 159 and/or user input features whereby the user can provide inputs to the system 100 to control all or portions of the operation of the system 100. The user input device 159 can include one or several keyboards, keypads, microphones, mouses, buttons, knobs, or the like. The interface 108 can be controlled by the one or several processors 166 to, for example, generate a graphical user interface (GUI) to provide information to the user.

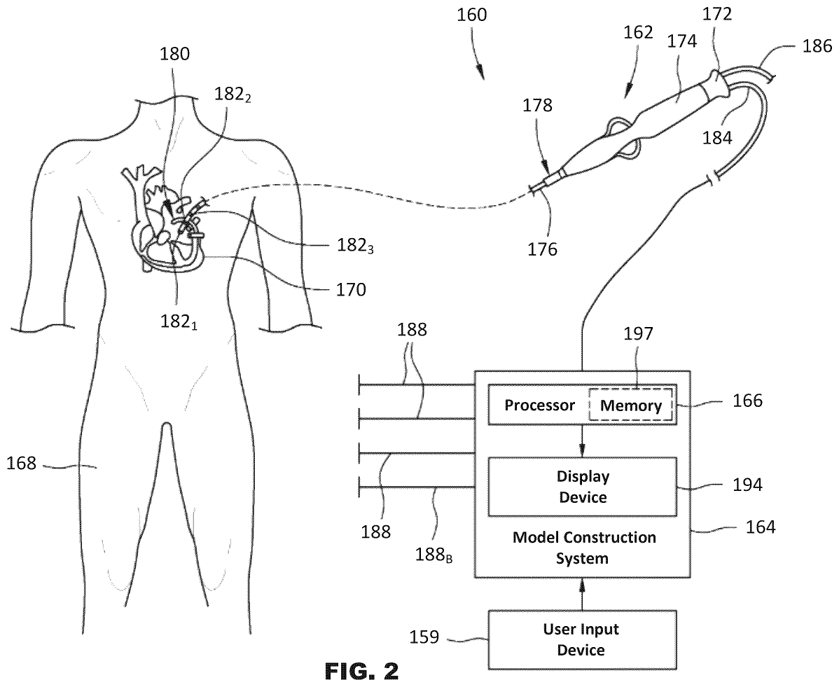

FIG. 2 illustrates a system 160 for generating surface models of cardiac structures. The system 100 can be system 160. More specifically, system 160 can be a subset of system 100. The system 160 can be used for generating surface models of cardiac structures. The system 160 includes, among other components, a medical device 162 and a model construction system 164. The medical device 162 can include a catheter, and the model construction system 164 can include, in part, the processing apparatus 166. The processing apparatus 166 may take the form of an electronic control unit, for example, which is configured to obtain information relating to the cardiac structure, and to generate a surface model of the cardiac structure using data generated via, for example, the catheter 162. The catheter 162 is configured to be inserted into a patient's body 168, and more particularly, into the patient's heart 170. The catheter 162 may include a cable connector or interface 172, a handle 174, a shaft 176 having a proximal end 178 and a distal end 180 and one or more sensors 182 (e.g., 182₁, 182₂, 182₃) mounted in or on the shaft 176 of the catheter 162. The sensors 182 can be disposed at or near the distal end 180 of the shaft 176. The connector 172 provides mechanical, fluid, and electrical connection(s) for cables, such as, for example, cables 184, 186 extending to the model construction system 164 and/or other components of the system 160 (e.g., a visualization, navigation, and/or mapping system (if separate and distinct from the model construction system 164), an ablation generator, irrigation source, etc.).

The sensors 182 can include one or several electrodes, contact sensors, pressure sensors, optical sensors, or the like. All the sensors 182 on the catheter 162 can be the same type of sensor or some or all the sensors 182 can be different types of sensors. For example, a plurality of the sensors 182 can include electrodes and at least one of the sensors can include a sensor configured to detect contact with tissue of the patient's heart. This sensor configured to detect contact with tissue of the patient's heart can include, for example, a proximity sensor, a force sensor, a pressure sensor, a piezoelectric transducer, a deflection sensor such as, for example, an optical deflection sensor, or the like. Examples of catheters including a force sensor include the Abbott TactiCath™, and Abbott TactiFlex™ ablation catheters. The catheter 162 can include a plurality of sensors configured to detect proximity and/or contact with the tissue of the patient's heart 170.

Contact with tissue of the patient's heart can be determined with the electrodes via, for example, one or several impedance measurements and specifically via one or several complex impedance measurements. Systems, devices, and methods in which complex impedance can be used to detect contact with tissue the patient's heart are described in U.S. Pat. No. 11,612,334, filed on Dec. 19, 2018, the entirety of which is hereby incorporated by reference herein.

The sensors 182 mounted in or on the shaft 176 of the catheter 162 are electrically connected to the model construction system 164, and the processing apparatus 166 thereof. The sensors 182 may be provided for a variety of diagnostic and therapeutic purposes including, for example and without limitation, EP studies, pacing, cardiac mapping, and ablation. One or more of the sensors 182 can be provided to perform a location or position sensing function. Accordingly, as the catheter 162 is moved along a surface of the cardiac structure and/or about the interior thereof, the sensor(s) 182 can be used to generate location data points that correspond to the surface of, or locations within, the cardiac structure. These location data points can then be used by, for example, the model construction system 164 in the generation of a geometry surface model of the cardiac structure.

The model construction system 164, and the processing apparatus 166 thereof, in particular, can be configured to receive data characterizing the patient's cardiac anatomy and to generate a surface model of the cardiac anatomy based in part on that received data. The processing apparatus 166 is configured to use, at least in part, data (location data and/or EP data/information) generated via the catheter 162 in the construction of one or both of a geometry surface model and an EP map.

The model construction system 164 is configured to acquire location data points generated via the sensor(s) 182 corresponding to the cardiac structure. The model construction system 164 is configured to then use those location data points in the construction and/or generation of the geometry surface model of the cardiac structure. The model construction system 164 can be configured to construct a geometry surface model, such as a faceted surface, based on some or all the generated location data points. In addition to constructing a geometry surface model of a structure, the model construction system 164 is configured to function with the sensor(s) 182 to generate location data points that are used in the construction of the geometry surface model. The model construction system 164 may include an electric field-based localization system, such as, for example, the EnSite NavX™ system and/or EnSite™ VoXel mode, and/or a magnetic-based localization system such as EnSite™ X EP, all commercially available from Abbott Laboratories, and generally shown with reference to U.S. Pat. No. 7,263,397 entitled “Method and Apparatus for Catheter Navigation and Location and Mapping in the Heart”, U.S. Pat. No. 10,328,308, entitled “Methods and System for Generating Electrophysiological Maps”, U.S. Pat. No. 10,758,137, entitled “Orientation Independent Sensing, Mapping, Interface and Analysis Systems and Methods”, U.S. Pat. No. 10,898,104, entitled “Field Concentrating Antennas for Magnetic Position Sensors”, U.S. Pat. No. 11,113,881, entitled “Systems and Methods for Generating a Three-dimensional Surface from a Segmented Volume”, U.S. Pat. No. 11,941,754, entitled “System and Method for Generating Three Dimensional Geometric Models of Anatomical Regions”, and U.S. Pat. No. 12,045,929, entitled “System and Method for Generating Three Dimensional Geometric Models of Anatomical Regions”, the entire disclosures of which is incorporated herein by reference. In other systems, devices, and computer-implemented methods in accordance with the present disclosure, however, the model construction system 164 may include other types of systems, such as, for example and without limitation: a magnetic-field based system such as the Carto™ System available from Biosense Webster, and as generally shown with reference to one or more of U.S. Pat. No. 6,498,944 entitled “Intrabody Measurement,” U.S. Pat. No. 6,788,967 entitled “Medical Diagnosis, Treatment and Imaging Systems,” and U.S. Pat. No. 6,690,963 entitled “System and Method for Determining the Location and Orientation of an Invasive Medical Instrument,” the entire disclosures of which are incorporated herein by reference, or the gMPS system from MediGuide Ltd., and as generally shown with reference to one or more of U.S. Pat. No. 6,233,476 entitled “Medical Positioning System,” U.S. Pat. No. 7,197,354 entitled “System for Determining the Position and Orientation of a Catheter,” and U.S. Pat. No. 7,386,339 entitled “Medical Imaging and Navigation System,” the entire disclosures of which are incorporated herein by reference; a combination electric field-based and magnetic field-based system such as the Carto 3™ System also available from Biosense Webster.

The sensor(s) 182 of the catheter 162 can include positioning sensors. The sensor(s) 182 produces signals indicative of catheter location (position and/or orientation) information. Where the model construction system 164 is an electric field-based system, the sensor(s) 182 may include one or more electrodes. In such a configuration, each of the electrodes may include one of a number of types of electrodes, such as, for example, tip electrodes, ring electrodes, button electrodes, coil electrodes, brush electrodes, flexible polymer electrodes, and spot electrodes. Alternatively, where the model construction system 164 is a magnetic field-based system, the sensor(s) 182 may include one or more magnetic sensors configured to detect one or more characteristics of a low-strength magnetic field. For instance, the sensor(s) 182 may include magnetic coils disposed on or in the shaft 176 of the catheter 162.

For purposes of clarity and illustration, the model construction system 164 will hereinafter be described as including an electric field-based system, such as, for example, the EnSite NavX™ system identified above. It will be appreciated that while the description below is primarily directed to describing systems, devices, and computer-implemented methods wherein the sensor(s) 182 includes one or more electrodes, the sensor(s) 182 may include one or more magnetic field sensors (e.g., coils). Accordingly, model construction systems that include positioning sensor(s) other than the sensors or electrodes described below remain within the spirit and scope of the present disclosure.

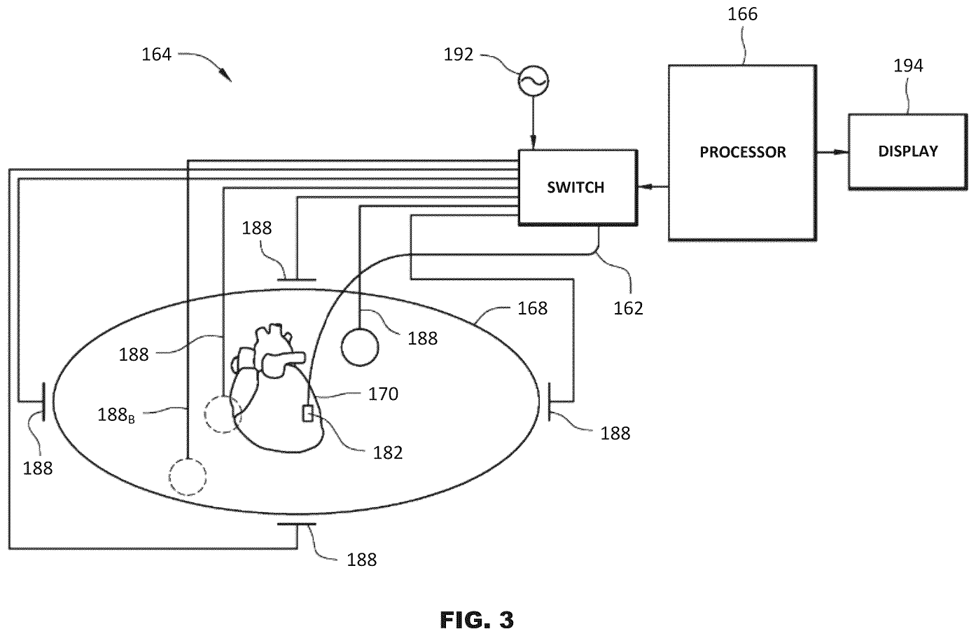

With reference to FIG. 3, a schematic depiction of portions of a system for generating surface models of cardiac structures is shown. In addition to the processing apparatus 166, the model construction system 164 may include, among other components, a plurality of patch electrodes 188, a multiplex switch 190, a signal generator 192, and a display device 194. Some or all these components, however, can be separate and distinct from the model construction system 164 but that are electrically connected to, and configured for communication with, the model construction system 164.

The processing apparatus 166 may include a programmable microprocessor or microcontroller or may include an application specific integrated circuit (ASIC). The processing apparatus 166 may include a central processing unit (CPU) and an input/output (I/O) interface through which the processing apparatus 166 may receive a plurality of input signals including, for example, signals generated by patch electrodes 188 and the sensor(s) 182, and generate a plurality of output signals including, for example, those used to control and/or provide data to, for example, the display device 194 and the switch 190. The processing apparatus 166 may be configured to perform various functions, such as those described in greater detail above and below, with appropriate programming instructions or code (i.e., software). Accordingly, the processing apparatus 166 is programmed with one or more computer programs encoded on a computer storage medium for performing the functionality described herein.

With the exception of the patch electrode 188B called a “belly patch,” the patch electrodes 188 are provided to generate electrical signals used, for example, in determining the position and orientation of the catheter 162. The patch electrodes 188 can be placed orthogonally on the surface of the body 168 and are used to create axes-specific electric fields within the body 168.

The sensor(s) 182 of the catheter 162 can be electrically coupled to the processing apparatus 166 and can be configured to serve a position sensing function. More particularly, the sensor(s) 182 are placed within electric fields created in the body 168 (e.g., within the heart) by exciting the patch electrodes 188. For purposes of clarity and illustration only, the description below will include describing systems, devices, and computer-implemented methods in which a single sensor 182 is placed within the electric fields. It will be appreciated, however, that a plurality of sensors 182 can be placed within the electric fields and then positions and orientations of each sensor can be determined using the techniques described below.

When disposed within the electric fields, the sensor 182 experiences voltages that are dependent on the location between the patch electrodes 188 and the position of the sensor 182 relative to tissue. Voltage measurement comparisons made between the sensor 182 and the patch electrodes 188 can be used to determine the location of the sensor 182 relative to the tissue. Accordingly, as the catheter 162 is swept about or along a particular area or surface of interest, the processing apparatus 166 receives signals (location information) from the sensor 182 reflecting changes in voltage levels on the sensor 182 and from the non-energized patch electrodes 188. Using various known algorithms, the processing apparatus 166 may then determine the location (position and orientation) of the sensor 182 and record it as a location data point corresponding to a location of the sensor 182 on the surface of, or within, the cardiac structure in a memory or storage device associated with, or accessible, by the processing apparatus 166, such as the memory 197. Prior to recording the location as a location data point, the raw location data represented by the signals received by the processing apparatus 166 may be corrected by the processing apparatus 166 to account for respiration, cardiac activity, and other artifacts using known or hereafter developed techniques. The system described in FIGS. 2and 3is further described in U.S. Pat. No. 8,909,502, entitled “Method and System for Constructing an Electrophysiology Map”, which is hereby incorporated by reference as though fully set forth herein.

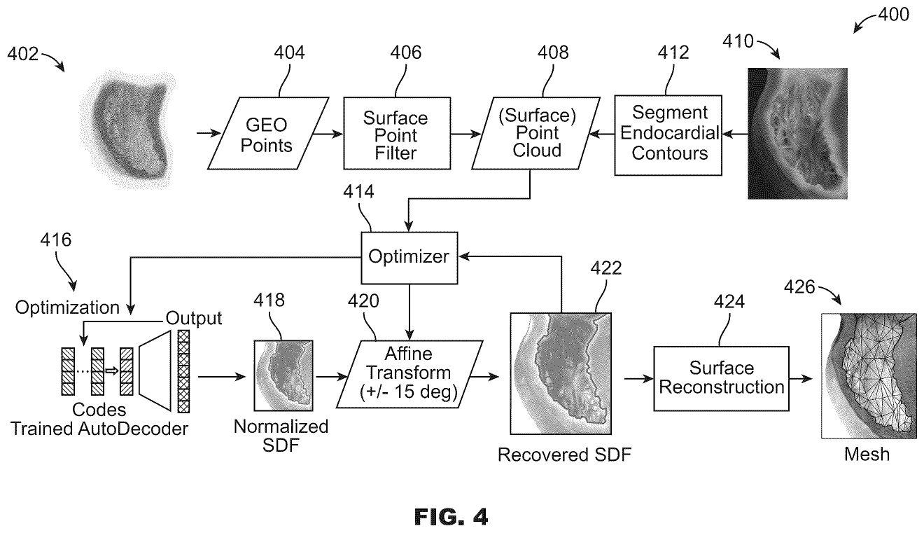

With reference now to FIG. 4, a schematic depiction of a process 400 for generating surface models of cardiac structures is shown. The process 400 can be performed by all or portions of system 100 and/or system 160. The process 400 can have two starting points depending on the type of data used, and specifically depending on the data gathering device 102 used to generate input data. For example, the process 400 can start with the insertion of a data gathering device 102 such as sensor 182 into the patient's heart and specifically into a cardiac chamber of the patient's heart. As indicated at step 402, this sensor can be used to physically map all or portions of the patient's heart and specifically to physically map all or portions of the chamber of the patient's heart to thereby generate a point cloud, for example a point cloud of GEO points at step 404. However, it will be appreciated that in some examples the data may have already been obtained (e.g., historically) and so the method does not require the data gathering device 102 to be inserted into the patient's heart.

The point cloud from step 404 can include both points on and off the surface of the patient's heart, and specifically on and off the surface of the chamber of the patient's heart. The GEO points can be filtered as indicated at step 406 to identify and/or select points located on the surface of the patient's heart and specifically located on the surface of the chamber of the patient's heart. The filtered surface points form a surface point cloud as indicated at step 408. In one approach, an approximating surface is defined that approximates the surface of the endocardium of the heart. The GEO points can be filtered to identify and/or select points located on the surface of the patient's heart based on proximity of the points to the approximating surface (e.g., points within a suitable distance, such as 5 mm, from the approximating surface). The approximating surface can be defined using any suitable approach such as, for example, using a convex hull algorithm.

Alternatively, the process 400 can start with the insertion of a data gathering device 102 such as an imaging device such as an echocardiograph and specifically an intracardiac echocardiograph. As indicated at step 410, this imaging device can image all or portions of the patient's heart and specifically can image all or portions of the chamber of the patient's heart, or in other words can generate an image such as via an echocardiograph and specifically an intracardiac echocardiograph. As indicated at step 412, the image can be segmented to identify contours in the 2D images and/or to identify surfaces in 3D images, and a surface point cloud can be created at step 408 by sampling from the identified contours and/or surfaces.

The process 400 proceeds to generating an optimized or final SDF as indicated in steps 414 through 422. This includes, at step 416, providing an input to a machine learning model, and specifically to a neural network, and generating a normalized SDF at step 418. The neural network can include an autoencoder, an autodecoder, a variational autoencoder (VAE), or the like.

The neural network can be a neural network that generates the normalize SDF 418 from an input “code” and an XYZ location. When provided an XYZ location, the neural network can produce a distance value for that location. The code selects for a particular surface which has been learned during training. The code can be associated with a surface from the training set; and/or can be a new code which “generates” a novel surface. Codes can be a high dimensional vector that specify a particular surface within the distribution of shape priors. A surface (in SDF format) for a particular code can be recovered by interrogating the neural network for XYZ locations that span the domain. The machined learning model can be trained to produce the normalized SDF at step 418 so that each coordinate of the XYZ location would be in the range of [−1.0, 1.0].

The initial code, also referred to herein as an initial input vector, can be selected and ingested into the neural network. This code can be selected In any desired manner, including, for example, selected at random, selected as representing the most typical cardiac geometry, or the like. The initial code can be used to produce a preliminary recovered SDF at step 422 (which is also referred to herein as a “preliminary SDF”).

The initial code can be optimized and specifically can be iteratively optimized to produce a series of recovered SDFs at step 422 until a final recovered SDF is produced at step 422 that best fits the surface point cloud from step 408. This can include optimizing the code to produce an optimized (final) recovered SDF at step 422 with minimal error as compared to the surface point cloud. This optimization can be performed using a variety of techniques including, for example, gradient descent, and more specifically including an Adaptive Moment Estimation (ADAM) optimizer. Use of the ADAM optimizer can assist in finding an absolute minimum in error as opposed to just a local minimum in error.

A gradient descent algorithm can be used to obtain the gradient of the error, or in other words, the direction of fastest descent at the given code, and then can take a step in that direction. This process can proceed until a minimum is found.

This optimization proceeds as follows, the initial code is selected and ingested into the neural network to generate a preliminary recovered SDF at step 422. The preliminary recovered SDF from step 422 is compared to points in the surface point cloud from step 408 to determine an error, or in other words, a difference between the preliminary recovered SDF from step 422 and the surface point cloud from step 408. Each point in the surface point cloud from step 408 can be compared to the preliminary SDF 422 from step 422 and the difference between that point in the surface point cloud from step 408 and the preliminary recovered SDF from step 422 can be the error value for that point. The absolute value of the errors for the points in the surface point cloud from step 408 as compared to the preliminary recovered SDF from step 422 can be aggregated. The absolute value of the errors for the points in the surface point cloud from step 408 can be combined to calculate a mean absolute error. The optimization can be performed (via repetition of steps 414 through 422) until the recovered SDF at step 422 produces a mean absolute error that is equal to or less than a specified maximum acceptable error.

As part of the optimization, an affine transform used in step 420 can also be optimized. The affine transform used in step 420 can be optimized on the recovered SDF from step 422 with respect to the point cloud from step 404 and/or with respect to the surface point cloud from step 408. Specifically, optimizing the affine transform used in step 420 can allow correction for error of fit arising due to skew, rotation, and/or scaling, which may arise due to a difference in relative orientation of the patient's heart and/or the chamber of the patient's heart with respect to the normalized SDF from step 418. The optimization of the affine transformation can be performed simultaneously with the optimization of the code, and the optimization of the affine transformation can be performed separate from the optimization of the code. Although the affine transform can be optimized simultaneous with the optimization of the code, such optimization may take more time to converge and/or may have a higher likelihood of getting caught in a local minimum due to the increased dimensionality of the search space arising from the combination of the code optimization and the affine transform optimization.

Where the affine transform used in step 420 is optimized separate from the optimization of the code, the optimization process can proceed iteratively wherein a code is selected and the affine transform for that code used in step 420 is then optimized. The optimization of the affine transform used in step 420 can include, for example, finding the rotation angles, scale factors, and/or skew that provide the best fit of the surface point cloud from step 408 to the recovered SDF from step 422 generated by that code. Once the optimal affine transform used at step 420 has been identified for that code, then the optimization can proceed to the next iterative step including determining the error for the recovered SDF at step 422 generated by that code and minimizing that error via application of gradient descent algorithm to identify a new code. This process can be iterated until the best code having the smallest error between the recovered SDF from step 422 and the surface point cloud from step 408 has been identified.

Optimizing the affine transform used at step 420 can be assisted by limiting the search space. Specifically, although one patient's heart may be rotated with respect to another patient's heart or with respect to the recovered SDF at step 422, the amount of this rotation is not limitless. Specifically, it has been found that these rotations of the patient's hearts are limited to a range of angles and can be limited to a range of angles of up to +/−15°. Thus, the search space of the affine transform used at step 420 can be constrained to a search space of +/−15°.

As a result of completion of the optimization, a final recovered SDF produced at step 422 (which is also referred to herein as “a final SDF”) is output for use in surface reconstruction at step 424. The final recovered SDF from step 422 is used to generate cardiac surfaces at step 424, which can be used to generate any suitable cardiac surface representation 426, such as a mesh that includes a faceted surface. The generation of the cardiac surface representation 426 at step 424 can include, for example, converting the final recovered SDF from step 422 into a voxel format by first creating a voxel grid of sufficient resolution spanning the volume covered by the final recovered SDF from step 422. The resolution of the voxel grid can be, for example, up to approximately 8 mm, up to approximately 6 mm, up to approximately 4 mm, less than approximately 3 mm, up to approximately 2 mm, approximately 2 mm, less than approximately 1 mm, approximately 1 mm, or any other or intermediate value. After the voxel grid has been created, each of the voxel cells in the voxel grid can then be populated with either a one or zero depending on the signed distance value of the final recovered SDF from step 422 at that location. For example, a location in the final recovered SDF from step 422 can have a negative value when that location is interior of the surface represented by the final recovered SDF from step 422 and a positive value when that location is exterior of the surface represented by the final recovered SDF from step 422. A voxel cell can be assigned a “1” label when that location has a negative value, and a voxel cell can be assigned a “0” label when that location has a positive value. Once all the cells have been assigned a value based on the final recovered SDF from step 422, then the final recovered SDF has been converted into a voxel representation in voxel format, at which point the voxel representation can be converted to the cardiac surface representation 426. The conversion into the cardiac surface representation 426 can include the generation of a mesh including a faceted surface such as a triangulated surface. Conversion from the voxel representation to the cardiac surface representation 426 can be performed via application of an algorithm such as the “Marching Cubes” algorithm.

Alternatively, the final recovered SDF can be used to generate the cardiac surface representation 426 by converting the SDF surface representation into a point cloud and then converting that point cloud into a faceted surface. The generation of this point cloud can include sampling the final recovered SDF to identify surface locations with the final recovered SDF and generating points in the point cloud corresponding to those sampled locations. The point cloud can be converted into the cardiac surface representation 426 via application of an algorithm such as, for example, an alpha shape algorithm, ball pivoting algorithm, a Poisson surface reconstruction algorithm, or the like.

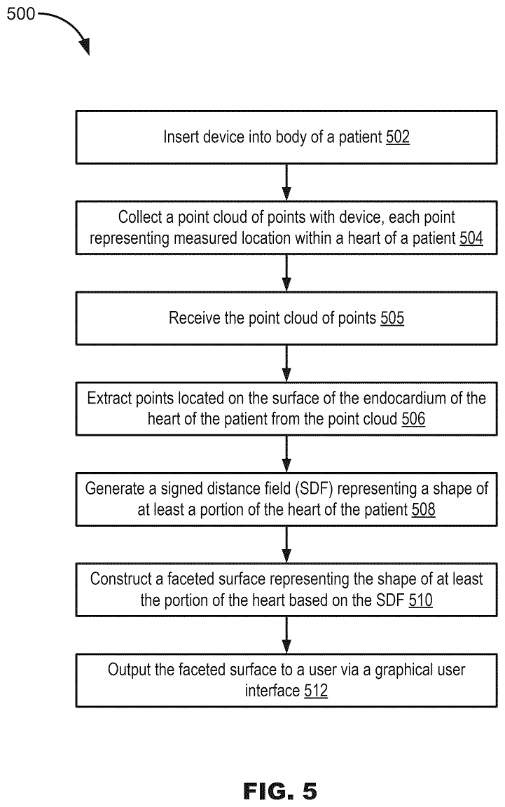

With reference now to FIG. 5, a flowchart illustrating a process 500 for generating surface models of cardiac structures is shown. The process 500 can be performed by system 100 and/or system 160. The process 500 can utilize a data gathering device 102 including at least one sensor 182. The process 500 can optionally begin with an optional step represented by block 502 wherein a medical device is inserted into the body of the patient. This medical device can include a catheter 162 including one or more sensors 182 on its distal end 180. The catheter 162, and specifically the distal end 180 of the catheter 162 can be advanced into the patient's heart 170 and specifically into a chamber of the patient's heart 170.

At an optional step represented by block 504, a point cloud of points is generated via the medical device, each of which corresponds to a measured location within the heart 170 of the patient. In addition to location data gathered for each point in the point cloud, the medical device can gather information indicative of whether the medical device, and specifically the sensor 182 on the medical device is contacting the patient's heart 170 or is in the blood pool in the patient's heart 170 without contacting the patient's heart 170. The contact information for a location can be associated with the location information, such that each point in the point cloud can include location information and associated contact information. For example, a point in the point cloud can include information identifying the location of the point and information indicating whether that point is on a surface of the patient's heart 170 and specifically on a surface of a chamber in the patient's heart 170. This information indicating whether a point is on a surface of the patient's heart can include a force, a pressure, an impedance, curvature, or the like.

Where optional steps 502 and 504 are not part of process 500, the process 500 can begin at block 505 wherein the point cloud of points is received. The point cloud of points can be received by a processor which can include one or more processors 166. The point cloud of points can be received by the one or more processors 166 from, for example, from the one or several data gathering devices 102 and/or one or more sensors 182. Some or all the following steps of process 500 can be performed by the one or more processors 166.

At block 506 points located on the surface of the endocardium of the patient's heart 170 are extracted from the point cloud. This can include filtering the points in the point cloud according to information indicating whether that point is on the surface of the patient's heart 170 or in other words is located on the surface of the endocardium of the patient's heart 170 or more specifically is located on the surface of the endocardium of a chamber of the patient's heart 170. Where each point includes location information paired and/or associated with information, such as contact information, indicating whether that point is on the surface of the patient's heart 170, the filtering can include comparing the information indicating whether that point is on the surface of the patient's heart 170 to one or several threshold values. For example, where the information indicating whether that point is on the surface of the patient's heart 170 includes a force, a pressure, and/or a curvature, the information indicating whether the point is on the surface of the patient's heart 170 can be compared to a threshold value for force, pressure, and/or curvature. If the information indicating whether the point is on the surface of the patient's heart 170 is less than the threshold value, then the point associated with that information can be identified as not on the surface of the patient's heart 170. Alternatively, and if the information indicating whether the point is on the surface of the patient's heart 170 is greater than the threshold value, then the point associated with that information can be identified as on the surface of the patient's heart 170. In the event that the information indicating whether the point is on the surface of the patient's heart 170 includes an impedance, then the impedance for a point can likewise be compared to a corresponding threshold value, and based on that comparison the point can be classified as either on the surface of the patient's heart 170 or off of the surface of the patient's heart 170. Alternatively, the points in the point cloud can be filtered via a convex hull algorithm.

Points extracted from the point cloud for being located on the surface of the endocardium can together form a surface point cloud.

At block 508 an SDF is generated based on the points in the surface point cloud and representing a shape of at least a portion of the patient's heart 170 and specifically representing a shape of the chamber of the patient's heart 170. The SDF can be generated based on the points of the surface point cloud and via machine learning model, and specifically via a neural network. Details of the generation of the SDF will be discussed at greater length below.

At block 510 faceted surface representing the shape of at least a portion of the heart is constructed based on the SDF generated in block 508. This can include converting the SDF to a voxel representation, then converting the voxel representation to a mesh forming a faceted surface and specifically forming a triangulated surface. The faceted surface can be constructed by converting the SDF to a point cloud and then converting the point cloud to a mesh forming a faceted surface and specifically forming a triangulated surface.

At block 512 the surface is output to the user via a graphical user interface. This can include controlling the display 194 via the one or more processors 166 to display the surface, and specifically the faceted surface to the user.

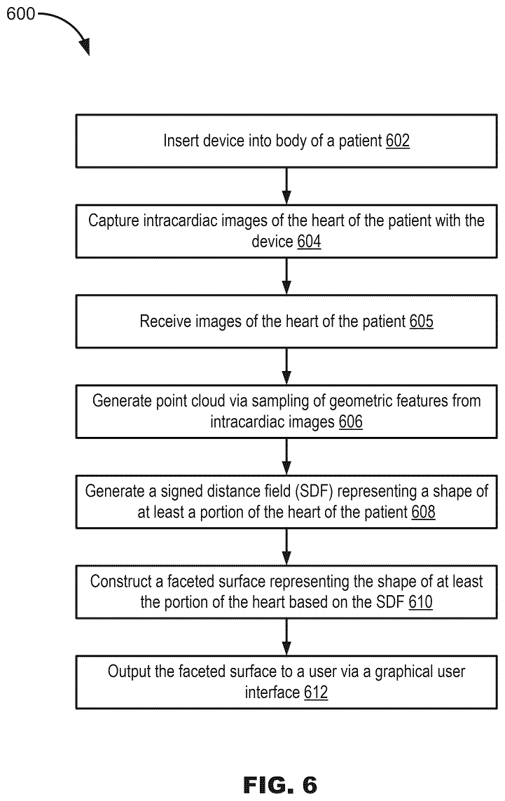

With reference now to FIG. 6, a flowchart illustrated the process 600 for cardiac surface generation with an imaging device is shown. The process 600 can be performed by system 100. The process 600 can utilize a data gathering device 102 including one or several imaging devices configured to generate images within the patient such as, for example, one or several echocardiographs and specifically one or several intracardiac echocardiographs, one or several cameras, or the like. The data gathering device 102 can generate one or more CT scans, MRI scans, or the like. Where the imaging device captured intracardiac images, the process 600 can optionally begin at an optional step represented by block 602 wherein a medical device is inserted into the body of the patient. This medical device can include a catheter 162 including one or more sensors 182, which sensors can include one or more imaging devices. The catheter 162, and specifically the distal end 180 of the catheter 162 can be advanced into the patient's heart 170 and specifically into a chamber of the patient's heart 170.

At an optional step represented by block 604 one or more image of the patient's heart are captured. These images can be intracardiac images of the patient's heart, and specifically of the chamber of the patient's heart. Alternatively, these images can be captured outside of the patient's body, and can be captured via, for example, a CT and/or MRI scan. These images can be captured with the medical device and specifically with the imaging devices of the medical device. These images can include one or more 2D images and/or one or more 3D images.

Where optional steps 602 and 604 are not part of process 600, the process 600 can begin at block 605 wherein the one or several images, and specifically the one or several intracardiac images of the heart of the patient are received. The images can be received by a processor which can include one or more processors 166. The images can be received by the one or more processors 166 from, for example, the one or several data gathering devices 102 and/or one or more sensors 182. Some or all the following steps of process 600 can be performed by the one or more processors 166.

At block 606 a point cloud is generated via sampling of geometric features from the intracardiac images. This can include, for example, segmenting the intracardiac images to identify contours in the 2D images and/or to identify surfaces in the 3D images. The contours and/or surfaces can be automatically identified via one or several image segmentation algorithms such as, for example, U-net or V-net DNN, active shape models, and/or the like. The segmentation algorithms can produce areas in the case of 2D images or produce volumes in the case of 3D images. A 3D surface point cloud can then be extracted from the contours and/or surfaces via sampling of the contours and/or surfaces.

A contour and/or a surface can include a variety of formats and can be converted to a point sampling from which the point cloud can be generated. A contour can include a series of connected line segments, and a surface can include a mesh. The contour and/or surface can be converted to a series of points from which the points of the point cloud can be extracted.

An alternative to using a contour or a surface to generate the point cloud, a segmented area and/or segmented volume can be used to generate the point cloud. For example, the segmented area and/or segmented volume can be converted to a series of points by identifying a bounding of the area and/or volume and sampling the boundary to identify points.

At block 608 an SDF is generated based on the points in the surface point cloud and represents a shape of at least a portion of the patient's heart 170 and specifically represents a shape of the chamber of the patient's heart 170. The SDF can be generated based on the points of the surface point cloud and via machine learning model, and specifically via a neural network. Details of the generation of the SDF will be discussed at greater length below.

At block 610 a faceted surface representing the shape of at least a portion of the heart is constructed based on the SDF generated in block 608. This can include converting the SDF to a voxel representation, then converting the voxel representation to a mesh forming a faceted surface and specifically forming a triangulated surface. The faceted surface can be constructed by converting the SDF to a point cloud and then converting the point cloud to a mesh forming a faceted surface and specifically forming a triangulated surface.

At block 612 the surface is output to the user via graphical user interface. This can include controlling the display 194 via the one or more processors 166 to display the surface, and specifically the faceted surface to the user.

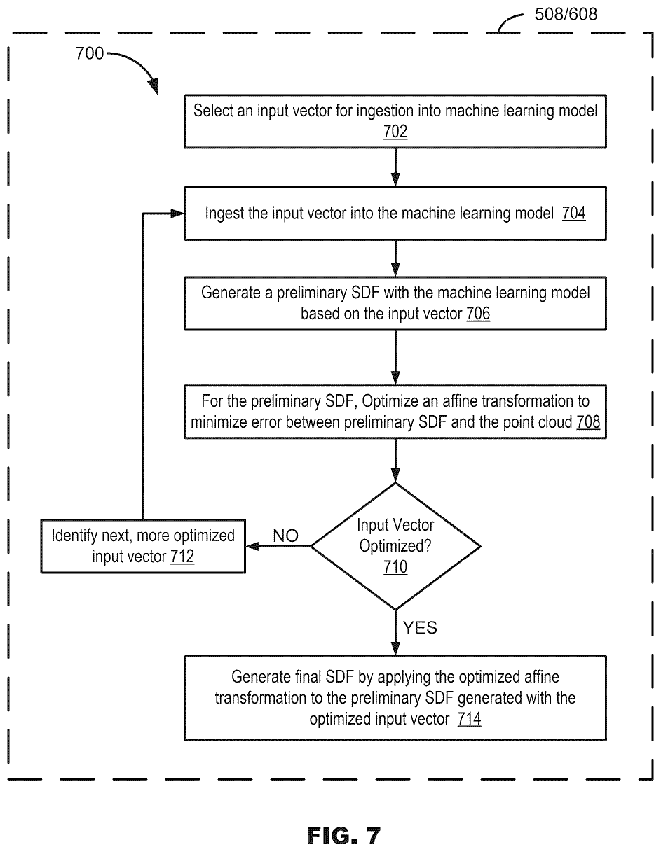

With reference now to FIG. 7, a flowchart illustrating a process 700 for generation of an SDF is shown. The process 700 can be performed as a part of, or in the place of the steps of blocks 508 and 608 of FIGS. 5 and 6 respectively. The process 700 begins at block 702, wherein an initial input vector (which is also referred to herein as an initial code) is selected for ingestion into the machine learning model, and specifically into the neural network. This initial input vector can be selected in any desired manner, including, for example, selected at random, selected as representing the most typical cardiac geometry, or the like. The initial input vectors can be initialized randomly at the beginning of training. The codes are learned along with the network weights. A typical geometry code can be found from the training set by finding the centroid of all the codes after training is completed.

At block 704, the input vector is ingested into the machine learning model and specifically is ingested into the neural network. At block 706 the machine learning model, and specifically the neural network generates a preliminary SDF based on the initial input vector.

At block 708, an affine transformation is optimized for the preliminary SDF to minimize error between the preliminary SDF and the point cloud. This can allow correction for errors of fit arising due to rotation of the patient's heart and/or the chamber of the patient's heart with respect to the generated SDF and/or allow for correction of scaling and/or size discrepancies between the point cloud and the preliminary SDF. The optimization of the affine transformation can correct for one or more of: skew, rotation, and scaling, or any combination thereof. This can include correcting for rotation and/or scaling of the preliminary SDF with respect to the point cloud.

The optimization of the affine transform can include, for example, finding the rotation angles, scale factors, and/or skew which provides the best fit of the point cloud to the preliminary SDF. Optimizing the affine transformation can be assisted by limiting the search space. Specifically, although one patient's heart may be rotated with respect to another patient's heart or with respect to the SDF, the amount of this rotation is not limitless. Specifically, it has been found that these rotations of the patient's hearts are limited to a range of angles and can be limited to a range of angles of up to +/−15°. Thus, the search space of the affine transform can be constrained to a search space of +/−15°. Once the optimal affine transform has been identified, then an error characterizing a difference between the optimized SDF and the point cloud can be generated.

At decision step 710, it is determined if the input vector is optimized. This can include determining if a more optimized input vector can be generated, which optimized input vector minimizes the error between the SDF created via ingestion of the associated input vector and the point cloud. This optimization can be performed using a variety of techniques including, for example, gradient descent, and more specifically including an ADAM optimizer.

Optimizing the input vector can include calculating an error value by, for example, comparing portions of the preliminary SDF to some or all of the points in the surface point cloud of block 506 or 606 to determine an error, or in other words, a difference between the preliminary SDF and the point cloud of block 506 or 606. Each point in the point cloud can be compared to the preliminary SDF and the difference between that point in the point cloud and the preliminary SDF can be the error value for that point. The absolute value of the errors for the points in the point cloud as compared to the preliminary SDF are aggregated. The absolute value of the errors for the points in the point cloud can be combined to calculate a mean absolute error.

As the optimization is performed, this error can be minimized resulting in the creation of an input vector that when ingested in by the machine learning model and specifically by the neural network creates an SDF closely matching the point cloud of block 506 or 606.

Determining if the input vector is optimized can include determining, according to gradient descent, and more specifically according to the ADAM optimizer, if the input vector can be further optimized. If it is determined that the input vector is not optimized, then the process 700 proceeds to block 712, wherein a next, more optimized input vector is identified. This next input vector can be identified according to gradient descent, and more specifically according to the ADAM optimizer. After the next, more optimized input vector is identified, then the process 700 returns to block 704 and proceeds as outlined above.

Returning again to decision state 710, if it is determined that the input vector is optimized, then the process 700 proceeds to block 714 and generates a final SDF. The final SDF can be generated by applying the optimized affine transformation to the preliminary SDF generated with the optimized input vector.

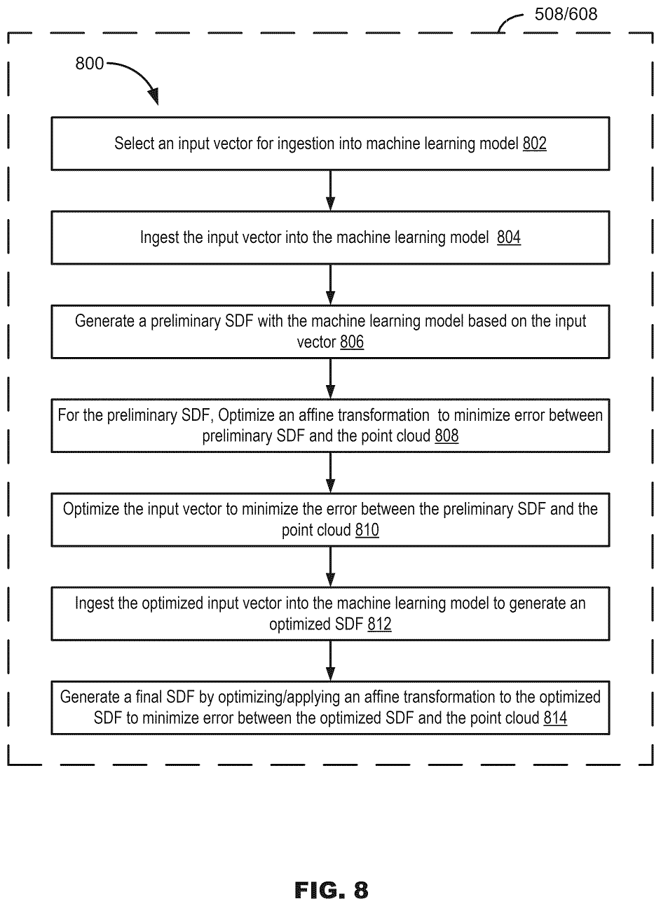

With reference now to FIG. 8, a flowchart illustrating a process 800 for generation of an SDF is shown. The process 800 can be performed as a part of, or in the place of the steps of blocks 508 and 608 of FIGS. 5 and 6 respectively. The process 800 begins at block 802, wherein an initial input vector is selected for ingestion into the machine learning model, and specifically into the neural network. This initial input vector can be selected in any desired manner, including, for example, selected at random, selected as representing the most typical cardiac geometry, or the like.

At block 804, the input vector is ingested into the machine learning model and specifically is ingested into the neural network. At block 806 the machine learning model, and specifically the neural network generates a preliminary SDF based on the initial input vector.

At block 808, an affine transformation is optimized for the preliminary SDF to minimize error between the preliminary SDF and the point cloud. This can allow correction for errors of fit arising due to rotation of the patient's heart and/or the chamber of the patient's heart with respect to the generated SDF and/or allow for correction of scaling and/or size discrepancies between the point cloud and the preliminary SDF. The optimization of the affine transformation can correct for one or more of: skew, rotation, and scaling, or any combination thereof. This can include correcting for rotation and/or scaling of the preliminary SDF with respect to the point cloud,

The optimization of the affine transform can include, for example, finding the rotation angles, scale factors, and/or skew which provides the best fit of the point cloud to the preliminary SDF. Optimizing the affine transformation can be assisted by limiting the search space. Specifically, although one patient's heart may be rotated with respect to another patient's heart or with respect to the SDF, the amount of this rotation is not limitless. Specifically, it has been found that these rotations of the patient's hearts are limited to a range of angles and can be limited to a range of angles of up to +/−15°. Thus, the search space of the affine transform can be constrained to a search space of +/−15°. Once the optimal affine transform has been identified, then an error characterizing a difference between the optimized SDF and the point cloud can be generated.

At block 810, the input vector is optimized to minimize the error between the preliminary SDF and the surface point cloud. Optimizing the input vector can result in the creation of an SDF with minimal error as compared to the point cloud of either block 506 or 606.

Optimizing the input vector can include calculating an error value by, for example, comparing portions of the preliminary SDF to some or all of the points in the surface point cloud of block 506 or 606 to determine an error, or in other words, a difference between the preliminary SDF and the point cloud of block 506 or 606. Each point in the point cloud can be compared to the preliminary SDF and the difference between that point in the point cloud and the preliminary SDF is the error value for that point. The absolute value of the errors for the points in the point cloud as compared to the preliminary SDF are aggregated. The absolute value of the errors for the points in the point cloud can be combined to calculate a mean absolute error.

As the optimization is performed, this error can be minimized resulting in the creation of an input vector that when ingested in by the machine learning model and specifically by the neural network creates an SDF closely matching the point cloud of block 506 or 606. The optimization of the affine transformation and the optimization of the input vector can be performed as part of a single optimization step or can be performed as separate optimization steps.

At block 812 the optimized input vector created by the optimizations is ingested into the machine learning model, and specifically into the neural network to generate an optimized SDF. At block 814 a final SDF is generated by optimizing/applying the affine transformation to the optimized SDF, the final SDF having the minimized and/or minimum error between the SDFs and the point cloud.

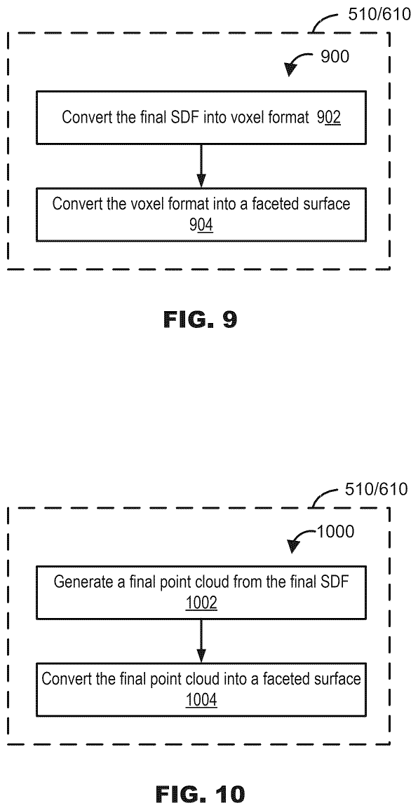

With reference now to FIG. 9, a flowchart illustrating a process 900 for constructing a surface representing at least a portion of the patient's heart 170 based on the SDF via a voxel representation is shown. The process 900 can be performed as a part of, or in the place of the steps of blocks 510 and 610 shown in FIGS. 5 and 6. The process begins at block 902, wherein the final SDF is converted into voxel format. This can include creating a voxel grid of sufficient resolution spanning the area and/or volume covered by the final SDF. The resolution of the voxel grid can be, for example, up to approximately 8 mm, up to approximately 6 mm, up to approximately 4 mm, up to approximately 3 mm, approximately 3 mm, up to approximately 2 mm, approximately 2 mm, up to approximately 1 mm, approximately 1 mm, or any other or intermediate value. After the voxel grid has been created, each of the voxel cells in the voxel grid can then be populated with either a one or zero depending on the signed distance value of the SDF at that location. For example, a location in the SDF can have a negative value when that location is interior of the surface represented by the SDF and a positive value when that location is exterior of the surface represented by the SDF. For example, a voxel cell can be assigned a “1” label when that location has a negative value, and a voxel cell can be assigned a “0” label when that location has a positive value. Once all the cells in the voxel grid have been assigned a value based on the SDF, then the SDF has been converted into a voxel representation in voxel format.

As indicated in step 904, after the SDF has been converted into a voxel representation in voxel format, then the voxel representation can be converted into a surface representation. The conversion into a surface representation can include the generation of a mesh including a faceted surface such as a triangulated surface. This conversion from the voxel representation to the surface representation can be performed via application of an algorithm such as the “Marching Cubes” algorithm.

With reference now to FIG. 10, a flowchart illustrating a process 1000 for constructing a surface representing the shape of at least a portion of the patient's heart 170 based on the SDF via a final point cloud is shown. The process 1000 be performed as a part of, or in the place of the steps of blocks 510 and 610 shown in FIGS. 5 and 6. The process begins at block 1002, wherein a final point cloud is generated from the final SDF, or in other words, the final SDF is converted into a final point cloud. The generation of this point cloud can include sampling the SDF to identify surface locations with the SDF and generating points in the point cloud corresponding to those sampled locations. 3D points can be sampled from the final SDF at locations in the final SDF having a distance near zero. This can include, selecting a location within the final SDF, determining the distance for that location, and comparing the distance of that location, and an absolute value of the distance of that location to a threshold value. This threshold value can be a distance and can be, for example, approximately 3 mm, less than approximately 3 mm, approximately 2 mm, less than approximately 2 mm, approximately 1 mm, less than approximately 1 mm, or any other or intermediate value. When the distance, and/or the absolute value of the distance at the selected location is less than the threshold value, then a point corresponding to that location can be added to the final point cloud.

At step 1004, the final point cloud can be converted into a surface representation. The conversion into a surface representation can include the generation of a mesh including a faceted surface such as a triangulated surface. The final point cloud can be converted into the surface representation via application of an algorithm such as, for example, an alpha shape algorithm, ball pivoting algorithm, a Poisson surface reconstruction algorithm, or the like.

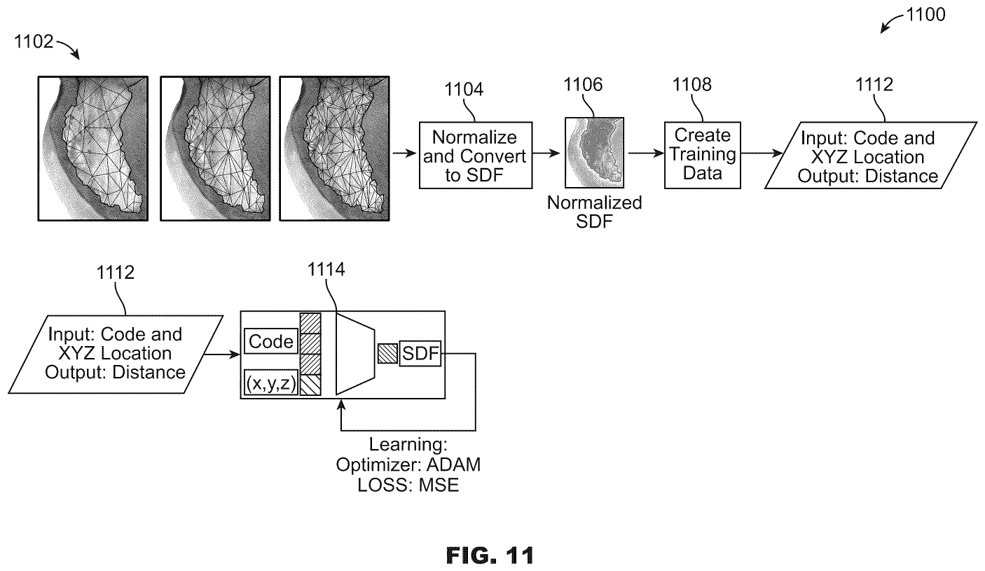

With reference now to FIG. 11, a schematic depiction of a process 1100 for training a machine learning model for generating surface models of cardiac structures is shown. The training can be performed with one or several processors 166 and memory 197. At step 1102, surface models, and more specifically, accurate surface models of patient hearts and specifically of cardiac chambers are generated. These surface models can be generated from segmented CT and/or MRI scans. These scans and/or images can be segmented as described above with respect to steps 604 and 606 of FIG. 6. The surface models can be created based on point clouds generated as described above with respect to steps 504 and 506 of FIG. 5.

At 1104, the surface models are normalized and converted into SDFs. The surface models can be normalized, or in other words, can be scaled, such that the mean distance (positive value) of all points in the cloud from the centroid is the same. These surface models can be normalized such that the mean distance (positive value) of all points in the cloud from the centroid is 1.0. The scale factor can be stored in memory 197 such that a mapping from the code to the scale factor can be learned by the machine learning model.

After the surface models are normalized, the normalized surface models can be converted into SDFs. An SDF can include a random dense sampling of XYZ points on the interior and exterior of the surface with the majority being very close to the surface. This dense sampling can include up to 10,000,000 points, up to 5,000,000 points, up to 1,000,000 points, up to 500,000 points, up to 250,000 points, approximately 250,000 points, or any other or intermediate number of points. At least 98% of points can be very close to the surface, at least 95% of points can be very close to the surface, at least 90% of points can be very close to the surface, at least 80% of points can be very close to the surface, at least 70% of points can be very close to the surface, or any other or intermediate percent of points can be very close to the surface from the surface model. A point can be classified as being very close to the surface when it is within a predetermined distance of the surface. This predetermined distance can be, for example, less than 5 mm, less than 3 mm, less than 2 mm, less than 1 mm, less than 0.5 mm, less than 0.3 mm, less than 0.2 mm, less than 0.1 mm, less than 0.05 mm, less than 0.03 mm, less than 0.02 mm, less than 0.01 mm, less than 0.005 mm, less than 0.001 mm, or less than any other or intermediated distance Having the majority of points close to the surface can improve training by training the machine learning model to be most accurate near the surface.

The format for representing a surface geometry as SDF is a list of points with associated signed distance values (x, y, z, d). A variety of computer-implemented methods can be used to compute the distance to surface for a given point, such as, for example, the method described in the paper “DeepSDF: Learning Continuous Signed Distance Functions for Shape Representation.” The SDF is depicted in 1106.

At 1108, code, which can be a unique code, is assigned to each surface model. The code can have any desired length, and the length can be determined by the size of the training set being created such that each piece surface model can have a code. The code can have a length of 32 characters.

The code can be randomly assigned or alternatively can be learned via an autoencoder such as, for example, a variational autoencoder (VAE). Learning of the code by a VAE can advantageously result in the creation of a normal distribution (Gaussian distribution) of the codes with similar SDFs having similar codes and proximate to each other in the space of codes.

The SDFs and their codes can be aggregated to form a set of training data indicated as 1112. The set of training data can have the format indicated in 1112, or as indicated below.

| | |
|---|---|
| Input | Output |

| | | | | |
|---|---|---|---|---|
| Single Sample | Code | XYZ | Scale (opt) | Distance |

A sample can be a point on a surface such as a point in the SDF. Thus, the total number of samples in the training set can be the sum of the number of points of each of the surfaces in the training set.

As indicated in 1114, the training set can be used to train the machine learning model. The machine learning model can be trained to generate an SDF based on an ingested code. The machine learning model can be a neural network. The neural network can include an autoencoder, an autodecoder, a VAE, or the like. The machine learning model can be trained by dividing the set of training data into a training set and a validation set. In some instances, approximately 20% of the data in the set of training data can be included in the validation set.

The machine learning model can be trained with the training according to any desired learning approach including, for example, backpropagation or particle swarm optimization. For example, the machine learning model training can employ a cost function (e.g., error of the output compared to ground truth), backpropagation (which can compute a gradient for all the weights that will reduce error), and optimization (to determine how much to alter the weight incorporating the gradient and often a momentum term). Weights can be updated via an optimization function such as ADAM utilizing a loss function that computes the discrepancy of the output SDF versus the ground truth. Backpropagation can be used to compute the weight gradient used by the ADAM optimizer. The machine learning model training can include the ingestion of a code into the machine learning model, generating an SDF with the machine learning model, calculating a mean absolute error between the SDF generated by the machine learning model and the SDF 1106. The machine learning model can then be trained and/or optimized according to the ADAM optimizer. The training of the machine learning model can be performed as described in the paper “DeepSDF: Learning Continuous Signed Distance Functions for Shape Representation.”

The following relate to numbered clauses of the disclosure:
1. A computer-implemented method of generating a surface model of a cardiac structure of a patient's heart, the computer-implemented method comprising:
   receiving, one or several processors, a point cloud comprising a plurality of points, wherein each of the points represents a measured location on a surface of the cardiac structure;
   extracting a surface point cloud from the point cloud, wherein the surface point cloud comprises points that represent locations on the surface of the cardiac structure;
   generating a final signed distance field (SDF) representing the surface of the cardiac structure via a machine learning model;
   constructing a surface model representing the surface of the cardiac structure based on the final SDF; and
   outputting or displaying the surface model to a user via a graphical user interface.
2. The computer-implemented method of clause 1, wherein the surface model comprises a faceted surface.
3. The computer-implemented method of clause 1 or 2, wherein contact information is associated with each of the points in the point cloud and indicates whether the represented location is on the surface of the cardiac structure.
4. The computer-implemented method of clause 3, wherein the contact information is generated based on at least one of:
   a contact force detected by a device used to measure locations used to generate the point cloud; or
   a complex impedance sensed by the device.
5. The computer-implemented method of clause 3 or 4, wherein the points are extracted from the point cloud based on the contact information.
6. The computer-implemented method of any one of clauses 1 to 5, further comprising defining an approximating surface that approximates the surface of the cardiac structure, and wherein the points are extracted from the point cloud based on proximity of the points to the approximating surface.
7. The computer-implemented method of any one of clauses 1 to 6, wherein the machine learning model comprises an autodecoder.
8. The computer-implemented method of any one of clauses 1 to 6, wherein the generation of the final SDF comprises:
   selecting an input vector for ingestion into the machine learning model; and
   determining an error between a preliminary SDF generated via the machine learning model and the surface point cloud.
9. The computer-implemented method of clause 8, wherein the generation of the final SDF comprises optimizing the input vector to minimize the error between the final SDF and the surface point cloud.
10. The computer-implemented method of clause 9, wherein the optimization of the input vector to minimize the error between the final SDF and the surface point cloud comprises optimizing the input vector via a gradient descent algorithm.
11. The computer-implemented method of clause 10, wherein the gradient descent algorithm comprises an Adaptive Moment Estimation (ADAM) optimizer.
12. The computer-implemented method of clause 10 or 11, further comprising optimizing an affine transformation used to compensate for an orientation of the patient's heart.
13. The computer-implemented method of clause 12, wherein the optimization of the affine transformation used to compensate for the orientation of the patient's heart is performed as part of optimizing the input vector to minimize the error between the final SDF and the surface point cloud.
14. The computer-implemented method of clause 13, further comprising generating the final SDF from the preliminary SDF by upscaling the preliminary SDF.
15. The computer-implemented method of clause 14, wherein the preliminary SDF is upscaled to the final SDF via application of a scale factor to the preliminary SDF.
16. The computer-implemented method of clause 15, wherein the construction of the surface model representing the surface of the cardiac structure based on the final SDF comprises:
   converting the final SDF into a voxel format; and
   converting the voxel format into the surface model.
17. The computer-implemented method of clause 15, wherein the construction of the surface model representing the surface of the cardiac structure based on the final SDF comprises:
   creating a final point cloud from the final SDF; and
   converting the final point cloud into the surface model.
18. The computer-implemented method of clause 17, wherein the final point cloud is converted into the surface model via application of at least one of:
   an alpha shape algorithm;
   a ball pivoting algorithm; and
   a Poisson surface reconstruction.
19. A tangible computer-readable storage medium storing instructions executable by the one or several processors (166) to cause the one or several processors (166) to accomplish the computer-implemented method of any one of clauses 1 to 18.
20. A computer-implemented method of generating a surface model of a cardiac structure of a patient's heart, the computer-implemented method comprising:
   receiving, by at least one processor, one or more intracardiac images of the cardiac structure;
   processing the one or more intracardiac images to generate a surface point cloud comprising points that represent locations on a surface of the cardiac structure;
   generating a final signed distance field (SDF) representing the surface of the cardiac structure via a machine learning model;
   constructing a surface model representing the surface of the cardiac structure based on the final SDF; and
   outputting or displaying the surface model to a user via a graphical user interface.
21. The computer-implemented method of clause 20, wherein the surface model comprises a faceted surface.
22. The computer-implemented method of any one of clauses 20 to 21, wherein the one or more intracardiac images are generated via intracardiac echocardiography.
23. The computer-implemented method of any one of clauses 20 to 22, wherein the processing of the one or more intracardiac images to generate the surface point cloud comprises one or more of:
   identifying a contour of the cardiac structure in the one or more intracardiac images and generating points representing locations on the identified contour of the cardiac structure; or
   identifying a surface of the cardiac structure in the one or more intracardiac images and generating points representing locations on the identified surface of the cardiac structure.
24. The computer-implemented method of any one of clauses 20 to 23, wherein the machine learning model comprises an autodecoder.
25. The computer-implemented method of any one of clauses 20 through 24, wherein the generation of the final SDF comprises:
   selecting an input vector for ingestion into the machine learning model; and
   determining an error between a preliminary SDF generated via the machine learning model and at least some of the points in the surface point cloud.
26. The computer-implemented method of clause 25, wherein the generation of the final SDF comprises optimizing the input vector to minimize the error between the final SDF and the at least some of the points in the surface point cloud.
27. The computer-implemented method of clause 26, wherein the optimization of the input vector to minimize the error between the final SDF and the at least some of the points in the surface point cloud comprises optimizing the input vector via a gradient descent algorithm.
28. The computer-implemented method of clause 27, wherein the gradient descent algorithm comprises an Adaptive Moment Estimation (ADAM) optimizer.
29. The computer-implemented method of any one of clauses 26 to 28, further comprising optimizing an affine transformation used to compensate for an orientation of the patient's heart.
30. The computer-implemented method of clause 29, wherein the optimization of the affine transformation is performed as part of optimizing the input vector to minimize the error between the final SDF and the at least some of the points in the surface point cloud.
31. The computer-implemented method of clause 29 or 30, wherein the preliminary SDF is normalized and further comprising generating the final SDF from the preliminary SDF by upscaling the preliminary SDF.
32. The computer-implemented method of clause 31, wherein the preliminary SDF is upscaled to the final SDF via application of a scale factor to the preliminary SDF.
33. The computer-implemented method of clause 32, wherein the construction of the surface model representing the surface of the cardiac structure based on the final SDF comprises:
   converting the final SDF into a voxel format; and
   converting the voxel format into the surface model.
34. The computer-implemented method of clause 32, wherein the construction of the surface model representing the surface of the cardiac structure based on the final SDF comprises:
   creating a final point cloud from the final SDF; and
   generating the surface model based on the final point cloud.
35. The computer-implemented method of clause 34, wherein the generation of the surface model based on the final point cloud is accomplished via application of at least one of: an alpha shape algorithm;
   a ball pivoting algorithm; and
   a Poisson surface reconstruction.
36. A tangible computer-readable storage medium storing instructions executable by the one or several processors (166) to cause the one or several processors (166) to accomplish the computer-implemented method of any one of clauses 20 to 35.
37. A system (160) for generating a surface model of a cardiac structure of a patient's heart, the system comprising:
   a display device (194);
   a medical device (162) comprising one or more sensors (182) configured to generate sensor output used to generate location data for points disposed on a surface of the cardiac structure and/or disposed within the cardiac structure; and
   a model reconstruction system (164) comprising one or several processors (166) and a tangible memory (197) storing non-transient instructions executable by the one or several processors to cause the one or several processors to:
      process the sensor output to generate a point cloud comprising a plurality of points, wherein each of the points represents a measured location on the surface of the cardiac structure and/or within the cardiac structure;
      extract a surface point cloud from the point cloud, wherein the surface point cloud comprises points that represent measured locations on the surface of the cardiac structure;
      generate a final signed distance field (SDF) representing a shape of a surface of the cardiac structure based at least in part of the surface point cloud via a machine learning model;
      construct a surface model representing the shape of the surface of the cardiac structure based on the final SDF; and
      output or display the surface model to a user via the display device (194).
38. The system (160) of clause 37, wherein the surface model comprises a faceted surface.
39. The system (160) of clause 37 or 38, wherein contact information is associated with each of the points in the point cloud and indicates whether the point represents a location on the surface of the cardiac structure.
40. The system (160) of clause 39, wherein the contact information is generated based on at least one of:
   a contact force detected by the medical device (162) used to generate the sensor output used to generate the location data for the points that represent measured locations on the surface of the cardiac structure; or
   a complex impedance sensed by the medical device (162).
41. The system (160) of clause 39 or 40, wherein the surface point cloud is extracted from the point cloud based on the contact information.
42. The system (160) of any one of clauses 37 to 41, wherein:
   the non-transient instructions are further executable by the one or several processors (166) to cause the one or several processors (166) to define an approximating surface that approximates the surface of the cardiac structure; and
   the surface point cloud is extracted from the point cloud based on proximity of the points to the approximating surface.
43. The system (160) of any one of clauses 37 to 42, wherein the machine learning model comprises an autodecoder.
44. The system (160) of any one of clauses 37 to 42, wherein the generation of the final SDF comprises:
   selecting an input vector for ingestion into the machine learning model; and
   determining an error between a preliminary SDF generated via the machine learning model and the surface point cloud.
45. The system (160) of clause 44, wherein the generation of the final SDF comprises optimizing the input vector to minimize the error between the final SDF and the surface point cloud.
46. The system (160) of clause 45, wherein the optimization of the input vector to minimize the error between the final SDF and the surface point cloud comprises optimizing the input vector via a gradient descent algorithm.
47. The system (160) of clause 46, wherein the gradient descent algorithm comprises an Adaptive Moment Estimation (ADAM) optimizer.
48. The system (160) of clause 46 or 47, wherein the non-transient instructions are further executable by the one or several processors to cause the one or several processors to optimize an affine transformation used to compensate for an orientation of the patient's heart.
49. The system (160) of clause 48, wherein the optimization of the affine transformation used to compensate for the orientation of the patient's heart is performed as part of optimizing the input vector to minimize the error between the final SDF and the surface point cloud.
50. The system (160) of clause 49, wherein the final SDF is generated from the preliminary SDF by upscaling the preliminary SDF.
51. The system (160) of clause 50, wherein the preliminary SDF is up scaled to the final SDF via application of a scale factor to the preliminary SDF.
52. The system (160) of clause 51, wherein the construction of the surface model representing the shape of the surface of the cardiac structure based on the final SDF comprises:
   converting the final SDF into a voxel format; and
   converting the voxel format into the surface model.
53. The system (160) of clause 51, wherein the construction of the surface model representing the shape of the surface of the cardiac structure based on the final SDF comprises:
   creating a final point cloud from the final SDF; and
   converting the final point cloud into the surface model.
54. The system (160) of clause 53, wherein the final point cloud is converted into the surface model via application of at least one of:
   an alpha shape algorithm;
   a ball pivoting algorithm; and
   a Poisson surface reconstruction.
55. A system (160) for generating a surface model of a cardiac structure of a patient's heart, the system comprising:
   a display device (194);
   a medical device (162) comprising one or more image sensors (182) configured to generate image sensor output used to generate surface data that defines a surface of the cardiac structure; and
   a model reconstruction system (164) comprising one or several processors (166) and a tangible memory (197) storing non-transient instructions executable by the one or several processors to cause the one or several processors to:
      process the image sensor output to generate the surface data that defines the surface of the cardiac structure;
      process the surface data to generate a surface point cloud comprising points representing locations on the surface of the cardiac structure;
      generate a final signed distance field (SDF) representing a shape of a surface of the cardiac structure based on the surface point cloud via a machine learning model;
      construct a surface model representing the shape of the surface of the cardiac structure based on the final SDF; and
      output or display the surface model to a user via the display device.
56. The system (160) of clause 55, wherein the surface model comprises a faceted surface.
57. The system (160) of any one of clauses 55 to 56, wherein the image sensor output is generated via intracardiac echocardiography.
58. The system (160) of any one of clauses 55 to 57, wherein the surface data that defines the surface of the cardiac structure comprises at least one of:
   a contour extracted from one or more 2-dimensional images of the cardiac structure;
   a surface extracted from one or more 3-dimensional images of the cardiac structure; or
   a point on a contour extracted from one or more 2-dimensional images of the cardiac structure.
59. The system (160) of any one of clauses 55 to 58, wherein the machine learning model comprises an autodecoder.
60. The system (160) of any one of clauses 55 through 59, wherein the generation of the final SDF comprises:
   selecting an input vector for ingestion into the machine learning model; and
   determining an error between a preliminary SDF generated via the machine learning model and at least some of the points in the surface point cloud.
61. The system (160) of clause 60, wherein the generation of the final SDF comprises optimizing the input vector to minimize the error between the final SDF and the at least some of the points in the surface point cloud.
62. The system (160) of clause 61, wherein the optimization of the input vector to minimize the error between the final SDF and the at least some of the points in the surface point cloud comprises optimizing the input vector via a gradient descent algorithm.
63. The system (160) of clause 62, wherein the gradient descent algorithm comprises an Adaptive Moment Estimation (ADAM) optimizer.
64. The system (160) of any one of clauses 61 to 63, wherein the non-transient instructions are further executable by the one or several processors to cause the one or several processors to optimize an affine transformation used to compensate for an orientation of the patient's heart.
65. The system (160) of clause 64, wherein the optimization of the affine transformation is performed as part of optimizing the input vector to minimize the error between the final SDF and the at least some of the points in the surface point cloud.
66. The system (160) of clause 64 or 65, wherein:
   the preliminary SDF is normalized; and
   wherein the non-transient instructions are further executable by the one or several processors to cause the one or several processors to generate the final SDF from the preliminary SDF by upscaling the preliminary SDF.
67. The system (160) of clause 66, wherein the preliminary SDF is upscaled to the final SDF via application of a scale factor to the preliminary SDF.
68. The system (160) of clause 67, wherein the construction of the surface model representing the surface of the cardiac structure based on the final SDF comprises:
   converting the final SDF into voxel format; and
   converting the voxel format into the surface model.
69. The system (160) of clause 67, wherein the construction of the surface model representing the shape of the surface of the cardiac structure based on the final SDF comprises:
   creating a final point cloud from the final SDF; and
   generating the surface model based on the final point cloud.
70. The system (160) of clause 69, wherein the final point cloud is converted into the surface model via application of at least one of:
   an alpha shape algorithm;
   a ball pivoting algorithm; and
   a Poisson surface reconstruction.

The methods, systems, and devices discussed above are examples. Some systems, devices, and/or methods of the present disclosure were described as processes depicted as flow diagrams or block diagrams. Although each may describe the operations as a sequential process, many of the operations can be performed in parallel or concurrently. In addition, the order of the operations may be rearranged. A process may have additional steps not included in the figure. Furthermore, the methods of the present disclosure may be implemented by hardware, software, firmware, middleware, microcode, hardware description languages, or any combination thereof. When implemented in software, firmware, middleware, or microcode, the program code or code segments to perform the associated tasks may be stored in a computer-readable medium such as a storage medium. Processors may perform the associated tasks.

It should be noted that the systems, devices, and methods discussed above are intended merely to be examples. It must be stressed that various systems, devices, and methods of the present disclosure may omit, substitute, or add various procedures or components as appropriate. Also, features described with respect to certain systems, devices, and methods of the present disclosure may be combined in various other systems, devices, and methods of the present disclosure. Different aspects and elements of the systems, devices, and methods of the present disclosure may be combined in a similar manner. Also, it should be emphasized that technology evolves and, thus, many of the elements are examples and should not be interpreted to limit the scope of the systems, devices, and methods of the present disclosure.

Specific details are given in the description to provide a thorough understanding of the systems, devices, and methods of the present disclosure. However, it will be understood by one of ordinary skill in the art that the systems, methods, and devices of the present disclosure may be practiced without these specific details. For example, well-known structures and techniques have been shown without unnecessary detail to avoid obscuring the systems, devices, and methods of the present disclosure. This description provides example systems, devices, and methods of the present disclosure only, and is not intended to limit the scope, applicability, or configuration of the systems, devices, and methods in accordance with the present disclosure. Rather, the preceding description of the systems, devices, and methods of the present disclosure will provide those skilled in the art with an enabling description for implementing systems, methods, and methods in accordance with the present disclosure. Various changes may be made in the function and arrangement of elements without departing from the spirit and scope of the systems, devices, and methods of the present disclosure.

The methods, systems, devices, graphs, and tables discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims. Additionally, the techniques discussed herein may provide differing results with different types of context awareness classifiers.

While illustrative and presently preferred examples of the disclosed systems, methods, and machine-readable media have been described in detail herein, it is to be understood that the inventive concepts may be otherwise variously embodied and employed, and that the appended claims are intended to be construed to include such variations, except as limited by the prior art.

Unless defined otherwise, all technical and scientific terms used herein have the same meaning as commonly or conventionally understood. As used herein, the articles “a” and “an” refer to one or to more than one (i.e., to at least one) of the grammatical object of the article. By way of example, “an element” means one element or more than one element. “About” and/or “approximately” as used herein when referring to a measurable value such as an amount, a temporal duration, and the like, encompasses variations of ±20% or ±10%, ±5%, or +0.1% from the specified value, as such variations are appropriate to in the context of the systems, devices, circuits, methods, and other implementations described herein. “Substantially” as used herein when referring to a measurable value such as an amount, a temporal duration, a physical attribute (such as frequency), and the like, also encompasses variations of ±20% or ±10%, ±5%, or +0.1% from the specified value, as such variations are appropriate to in the context of the systems, devices, circuits, methods, and other implementations described herein. As used herein, including in the claims, “and” as used in a list of items prefaced by “at least one of” or “one or more of” indicates that any combination of the listed items may be used. For example, a list of “at least one of A, B, and C” includes any of the combinations A or B or C or AB or AC or BC and/or ABC (i.e., A, B, and C). Furthermore, to the extent more than one occurrence or use of the items A, B, or C is possible, multiple uses of A, B, and/or C may form part of the contemplated combinations. For example, a list of “at least one of A, B, and C” may also include AA, AAB, AAA, BB, etc.

Having described several systems, devices, and methods of the present disclosure, it will be recognized by those of skill in the art that various modifications, alternative constructions, and equivalents may be used without departing from the spirit of the systems, devices, and methods of the present disclosure. For example, the above elements may merely be a component of a larger system, wherein other rules may take precedence over or otherwise modify the application of the systems, devices, and methods of the present disclosure. Also, a number of steps may be undertaken before, during, or after the above elements are considered. Accordingly, the above description should not be taken as limiting the scope of the system, devices, and methods of the present disclosure.

Also, the words “comprise”, “comprising”, “contains”, “containing”, “include”, “including”, and “includes”, when used in this specification and in the following claims, are intended to specify the presence of stated features, integers, components, or steps, but they do not preclude the presence or addition of one or more other features, integers, components, steps, acts, or groups.

1-36. (canceled)

37. A system for generating a surface model of a cardiac structure of a patient's heart, the system comprising:
a display device;
a medical device comprising one or more sensors configured to generate sensor output used to generate location data for points disposed on a surface of the cardiac structure and/or disposed within the cardiac structure; and
a model reconstruction system comprising one or several processors and a tangible memory storing non-transient instructions executable by the one or several processors to cause the one or several processors to:
process the sensor output to generate a point cloud comprising a plurality of points, wherein each of the points represents a measured location on the surface of the cardiac structure and/or within the cardiac structure;
extract a surface point cloud from the point cloud, wherein the surface point cloud comprises points that represent measured locations on the surface of the cardiac structure;
generate a final signed distance field (SDF) representing a shape of a surface of the cardiac structure based at least in part of the surface point cloud via a machine learning model;
construct a surface model representing the shape of the surface of the cardiac structure based on the final SDF; and
output or display the surface model to a user via the display device (194).

38. The system of claim 37, wherein the surface model comprises a faceted surface.

39. The system of claim 37, wherein contact information is associated with each of the points in the point cloud and indicates whether the point represents a location on the surface of the cardiac structure.

40. The system of claim 39, wherein the contact information is generated based on at least one of:
a contact force detected by the medical device used to generate the sensor output used to generate the location data for the points that represent measured locations on the surface of the cardiac structure; or
a complex impedance sensed by the medical device.

41. The system of claim 39, wherein the surface point cloud is extracted from the point cloud based on the contact information.

42. The system of claim 37, wherein:
the non-transient instructions are further executable by the one or several processors to cause the one or several processors to define an approximating surface that approximates the surface of the cardiac structure; and
the surface point cloud is extracted from the point cloud based on proximity of the points to the approximating surface.

43. The system of claim 37, wherein the machine learning model comprises an autodecoder.

44. The system of claim 37, wherein the generation of the final SDF comprises:
selecting an input vector for ingestion into the machine learning model; and
determining an error between a preliminary SDF generated via the machine learning model and the surface point cloud.

45. The system of claim 44, wherein the generation of the final SDF comprises optimizing the input vector to minimize the error between the final SDF and the surface point cloud.

46. The system of claim 45, wherein the optimization of the input vector to minimize the error between the final SDF and the surface point cloud comprises optimizing the input vector via a gradient descent algorithm.

47. The system of claim 46, wherein the gradient descent algorithm comprises an Adaptive Moment Estimation (ADAM) optimizer.

48. The system of claim 46, wherein the non-transient instructions are further executable by the one or several processors to cause the one or several processors to optimize an affine transformation used to compensate for an orientation of the patient's heart.

49. The system of claim 48, wherein the optimization of the affine transformation used to compensate for the orientation of the patient's heart is performed as part of optimizing the input vector to minimize the error between the final SDF and the surface point cloud.

50. The system of claim 49, wherein the final SDF is generated from the preliminary SDF by upscaling the preliminary SDF.

51. The system of claim 50, wherein the preliminary SDF is up scaled to the final SDF via application of a scale factor to the preliminary SDF.

52. The system of claim 51, wherein the construction of the surface model representing the shape of the surface of the cardiac structure based on the final SDF comprises:
converting the final SDF into a voxel format; and
converting the voxel format into the surface model.

53. The system of claim 51, wherein the construction of the surface model representing the shape of the surface of the cardiac structure based on the final SDF comprises:
creating a final point cloud from the final SDF; and
converting the final point cloud into the surface model.

54. The system of claim 53, wherein the final point cloud is converted into the surface model via application of at least one of:
an alpha shape algorithm;
a ball pivoting algorithm; and
a Poisson surface reconstruction.

55-70. (canceled)
